(19) United States
(12) Patent Application Publication
(10) Pub. No.: US 2024/0041639 A1
(43) Pub. Date: Feb. 8, 2024

(54) AT LEAST ONE TUBE, FLUID COLLECTION SYSTEMS INCLUDING THE SAME, AND METHODS OF USING THE SAME

(71) Applicant: PUREWICK CORPORATION, Covington, GA (US)

(72) Inventors: Justin Wolfe, Lawrenceville, GA (US); Ashely Marie Johannes, Statham, GA (US)

(21) Appl. No.: 18/265,736

(22) PCT Filed: Dec. 8, 2021
(86) PCT No.: PCT/US2021/062440

§ 371 Date: Jun. 7, 2023

Related U.S. Application Data
(60) Provisional application No. 63/124,271, filed on Dec. 11, 2020.

(51) Int. Cl. A61F 5/455; A61F 5/453

(52) U.S. Cl. CPC A61F 5/455; A61F 5/453

(57) ABSTRACT

An example tube with an elongated cross-section includes an inlet, an outlet downstream from the inlet, and an intermediate portion between the inlet and the outlet. The tube exhibits an elongated cross-section because at least the intermediate portion exhibits a width that is significantly greater than the thickness thereof. The elongated cross-section of the intermediate portion relative to its width allows the tube to be at least one of more flexible than or allows the intermediate portion to be positioned underneath a patient while minimizing the risk of bed sores relative to at least some conventional tubes.

### CROSS-REFERENCE TO RELATED APPLICATIONS

This application claims priority to U.S. Provisional Patent Application No. 63/124,271 filed on Dec. 11, 2020, the disclosure of which is incorporated herein, in its entirety, by this reference.

### BACKGROUND

A patient may have limited or impaired mobility such that typical urination processes are challenging or impossible. For example, the patient may have surgery or a disability that impairs mobility. In another example, the patient may have restricted travel conditions such as those experience by pilots, drivers, and workers in hazardous areas. Additionally, fluid collection from the patient may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of fluid collection assemblies continue to seek new and improved devices, systems, and methods to collect urine.

### SUMMARY

Embodiments disclosed herein include tubes with an elongated cross-section configured to be attached, either directly or indirectly, to a fluid collection assembly, fluid collection systems including the same, and methods of using the same. In an embodiment, a tube configured to be connected to a fluid collection assembly is disclosed. The tube including an inlet exhibiting an inlet width and an inlet thickness that are each measured perpendicular to a longitudinal axis of the tube. The inlet width is at most about 4 times greater than the inlet thickness. The tube includes an outlet downstream from the inlet. The outlet exhibiting an outlet width and an outlet thickness that are each measured perpendicular to the longitudinal axis of the tube. The tube includes an intermediate portion between the inlet and the outlet. The intermediate portion exhibits an intermediate width and an intermediate thickness that are each measured perpendicular to the longitudinal axis of the tube. The intermediate width is at least about 5 times greater than the intermediate thickness. The inlet, the outlet, and the intermediate portion define a conduit extending from the inlet to the outlet.

In an embodiment, a fluid collection system is disclosed. The fluid collection system includes a fluid collection assembly defining a fluid outlet, a fluid storage container defining a fluid inlet, and at least one tube configured to be connected to the fluid collection assembly. The at least one tube comprising an inlet, an outlet downstream from the inlet, and an intermediate portion between the inlet and the outlet. The intermediate portion exhibiting an intermediate width and an intermediate thickness that are each measured perpendicular to the longitudinal axis of the tube. The intermediate width at least about 5 times greater than the intermediate thickness. The inlet, the outlet, and the intermediate portion define a conduit extending from the inlet to the outlet.

In an embodiment, a method of using a fluid collection system is disclosed. The method includes receiving one or more bodily fluids from a patient with a fluid collection assembly, the fluid collection assembly including a fluid outlet and flowing the one or more bodily fluids out of the fluid outlet, into an inlet of a tube, through an intermediate portion of the tube, and out an outlet of the tube. The inlet exhibits an inlet width and an inlet thickness that are each measured perpendicular to a longitudinal axis of the tube. The inlet width is at most about 4 times greater than the inlet thickness. The outlet located downstream from the inlet. The outlet exhibits an outlet width and an outlet thickness that are each measured perpendicular to the longitudinal axis of the tube. The intermediate portion is between the inlet and the outlet. The intermediate portion exhibits an intermediate width and an intermediate thickness that are each measured perpendicular to the longitudinal axis of the tube. The intermediate width is at least about 5 times greater than the intermediate thickness. The inlet, the outlet, and the intermediate portion define a conduit extending from the inlet to the outlet.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a tube with an elongated cross-section, according to an embodiment.

FIG. 1B is a cross-sectional view of the tube taken along plane 1B-1B shown in FIG. 1A, according to an embodiment.

FIG. 2A is an isometric view of a tube with an elongated cross-section, according to an embodiment.

FIG. 2B is a cross-sectional schematic of the tube taken along plane 2B-2B shown in FIG. 2A, according to an embodiment.

FIG. 3A is an isometric view of a tube with an elongated cross-section that is formed from a plurality of distinct components that are attached together, according to an embodiment.

FIG. 3B is a cross-sectional schematic of the tube taken along plane 3B-3B as shown in FIG. 3A, according to an embodiment.

FIGS. 4-6 are cross-sectional schematics of different tubes with elongated cross-sections that include different fluid flow features, according to different embodiments.

FIG. 7 is a cross-sectional schematic of a portion of a tube, according to an embodiment.

FIG. 8A is a cross-sectional schematic of a tube having an elongated cross-section, according to an embodiment.

FIG. 8B is a cross-sectional schematic of a tube having an elongated cross-section, according to an embodiment.

FIG. 9A is an isometric view of fluid collection system that includes a female fluid collection assembly connected to a tube, according to an embodiment.

FIGS. 9B and 9C are cross-sectional schematics of the female fluid collection system taken along planes 9B-9B and 9C-9C as shown in FIG. 9A, according to an embodiment.

FIG. 10A is an isometric view of a portion of a fluid collection system that includes a male fluid collection assembly and a tube, according to an embodiment.

FIG. 10B is a cross-sectional view of the fluid collection system taken along plane 10B-10B as shown in FIG. 10A, according to an embodiment.

FIG. 11 is a block diagram of a fluid collection system, according to an embodiment.

### DETAILED DESCRIPTION

Embodiments disclosed herein include tubes with an elongated cross-section configured to be attached, either directly or indirectly, to a fluid collection assembly, fluid collection systems including the same, and methods of using the same. An example tube with an elongated cross-section includes an inlet, an outlet downstream from the inlet, and an intermediate portion between the inlet and the outlet. The tube exhibits an elongated cross-section because at least the intermediate portion exhibits a width that is significantly greater than the thickness thereof. The elongated cross-section of the intermediate portion allows the tube to be at least one of more flexible than or allows the intermediate portion to be positioned underneath a patient (i.e., an individual using a fluid collection assembly) while minimizing the risk of bed sores relative to at least some conventional tubes.

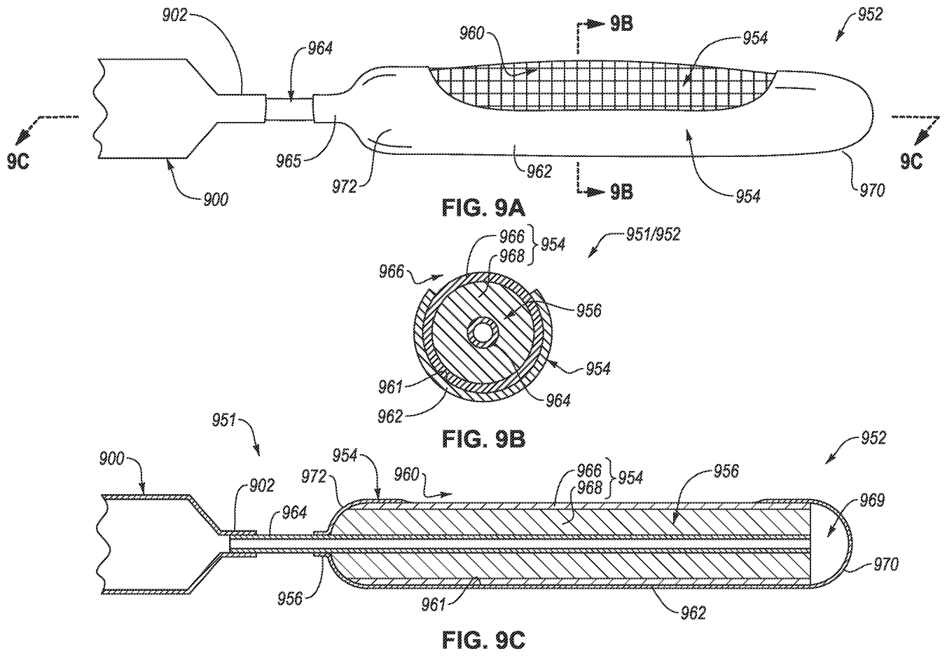

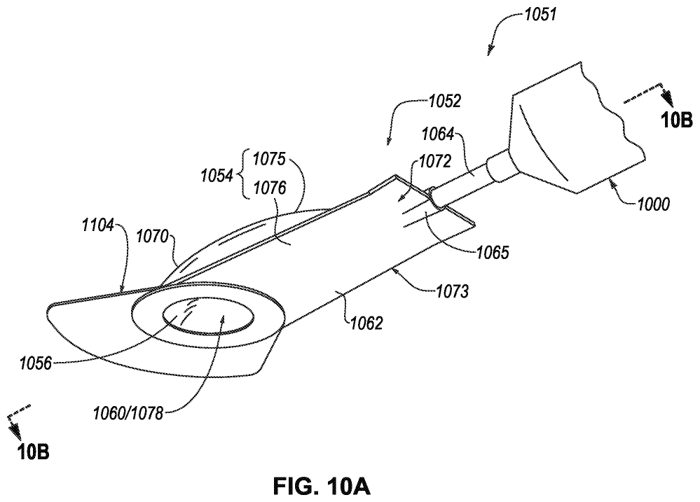

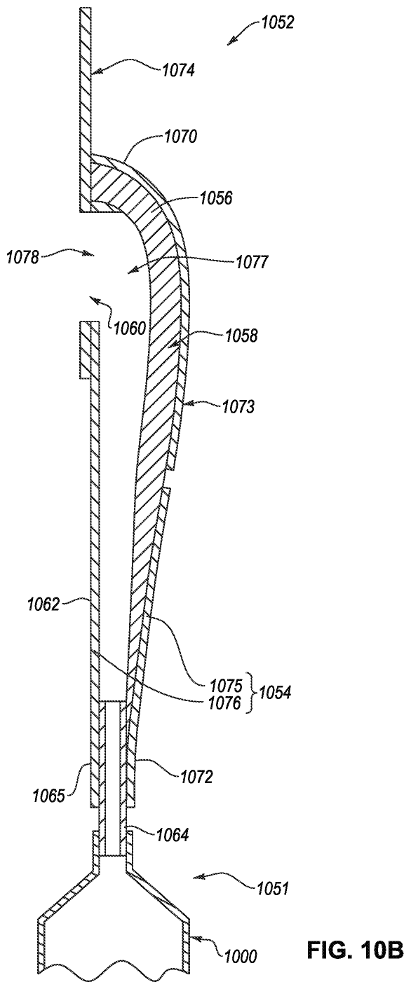

For example, the inlet of the tube with an elongated cross-section may be attached, either directly or indirectly, to a fluid collection assembly to form a fluid collection system. The fluid collection assembly may include a female fluid collection assembly (FIGS. 9A-9C), a male fluid collection assembly (FIGS. 10A and 10B), or any other suitable fluid collection assembly (e.g., a Foley catheter). During use, the fluid collection assembly may receive one or more bodily fluids from a patient. The bodily fluids may include urine, blood, semen, sweat, or any other bodily fluids that may be discharged from the urethral opening or the region about the urethral opening of the patient. The bodily fluids received by the fluid collection assembly may flow through the fluid collection assembly to a fluid outlet thereof. The fluid outlet may be attached to the inlet of the tube. As such, the bodily fluids may flow out of the fluid outlet of the fluid collection assembly and into the tube via the inlet of the tube. The bodily fluids may flow through the tube to the outlet. The bodily fluids may flow out of the outlet and, for example, into a fluid storage container that is connected, either directly or indirectly, to the outlet of the tube.

The elongated cross-section (i.e., relative large width relative to the thickness) of the intermediate portion of the tubes is an improvement over at least some conventional tubes that exhibit a generally circular cross-sectional shape or any other non-elongated cross-section (“conventional tubes”). In an example, the conventional tubes are less likely or unable to exhibit sharp bends therein without kinking. Kinking the conventional tubes prevents the bodily fluids from flowing through the conventional tube and, when the fluid collection system includes a vacuum source, prevents the vacuum source from removing the bodily fluids from the fluid collection assembly. As such, kinking the conventional tubes may inhibit the correct functioning of the fluid collection system that includes the conventional tubes. However, the elongated cross-section of the intermediate portion of the tubes causes the intermediate portion to exhibit high flexibility that allows the intermediate portion of the tubes disclosed herein to be bent sharply substantially without kinking. In an example, the vertical profile of the intermediate portion of the tubes with an elongated cross-section may be less than the vertical profile of the conventional tubes since the thickness of the intermediate portion is significantly less than the width thereof. The smaller vertical profile of the intermediate portion of the tubes with an elongated cross-section allows the intermediate portion to be positioned underneath the patient while being less likely to cause bed sores compared to the conventional tubes. For instance, the conventional tubes may need to curve upward to go over the thigh or other anatomical feature of the patient since the conventional tubes cannot be positioned under the thighs or other anatomical feature of the patient due to the risk of bed sores. The upward curvature of the conventional tube may inhibit the flow of bodily fluids therein since the bodily fluids must flow against gravity to flow through the upward curvature. However, unlike to conventional tube, the intermediate portion of the tubes with an elongated cross-section may be positioned under the thigh or other anatomical feature of the patient due to the smaller vertical profile thereof compared to the conventional tube. The elongated cross-section of the intermediate portion also may allow the tube to distribute a force applied therefrom to a larger surface area of the patient than a conventional tube which reduces the risk of bed sores.

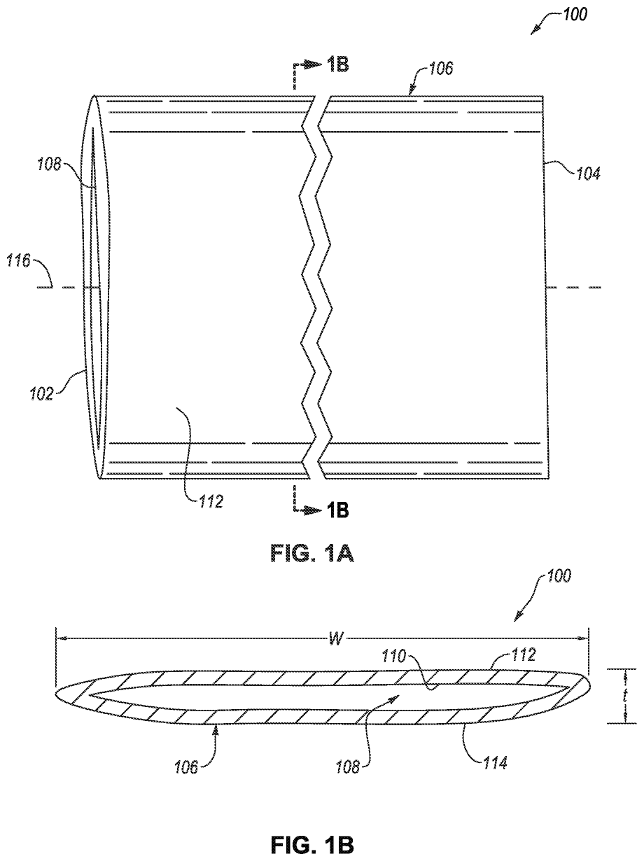

FIG. 1A is an isometric view of a tube 100 with an elongated cross-section, according to an embodiment. The break in the illustrated tube 100 indicates that the tube 100 may have any suitable length. FIG. 1B is a cross-sectional view of the tube 100 taken along plane 1B-1B shown in FIG. 1A, according to an embodiment. The tube 100 includes an inlet 102 and an outlet 104 downstream from the inlet 102. The tube 100 also includes an intermediate portion 106 between the inlet 102 and the outlet 104. The inlet 102, the outlet 104, and the intermediate portion 106 may define a conduit 108 through which one or more bodily fluids may flow. For example, the inlet 102, the outlet 104, and the intermediate portion 106 may include at least one inner surface 110 that defines the conduit 108.

The tube 100 includes a first outer surface 112 and a second outer surface 114 opposite the first outer surface 112. When the tube 100 is resting on a surface, the first outer surface 112 may be generally parallel to the second outer surface 114 thereby allowing the tube 100 to lay substantially flat. The first and second surfaces 112, 114 may be separated by a fold in the tube 100. In an embodiment, the tube 100 may include a feature that is configured to preferentially fold, such as a seam or a crease. The feature that is configured to preferentially fold causes the same portions of the tube 100 to generally form the first and second outer surfaces 112, 114. This may allow markings, such as marking indicating length, to be formed on one or more of the first or second outer surfaces 112, 114 with the expectation that the markings will face an individual (e.g., a medical practitioner, patient, etc.). However, it is noted that the tube 100 may be folded at locations other than the locations that are configured to preferentially fold due the high flexibility of the tube 100. In an embodiment, the tube 100 does not include the features that are configured to fold which may make manufacturing the tube 100 easier since such features do not need to be formed on the tube 100. However, the portions of the tube 100 that the form the first and second outer surfaces 112, 114 may vary since the location of the fold may also vary. Also, the tube 100 may be unable to form crisps folds when the tube 100 does not include the features that are configured to preferentially fold which may increase the vertical profile (e.g., thickness) of the tube 100.

The tube 100 may be formed from one or more panels. In an embodiment, the tube 100 is formed from a single panel. In other words, the first and second outer surfaces 112, 114 of the tube 100 may be formed from the same panel. In an example, the tube 100 formed from the single panel may be formed by a blown film extrusion process or other extrusion process. In an example, the tube 100 formed from the single panel may be formed by folding the panel in half and attaching together the edges of the panel opposite the fold (e.g., via ultrasonic welding, radio frequency welding, an adhesive, etc.). In such an example, the edges of the single panel that are attached together may form a feature that is configured to preferentially fold. In an example, the tube 100 may be formed in a generally tube-like (e.g., hollow cylindrical) shape (e.g., using an extrusion process) such that there are no edges of the panel that need to be attached together to form the tube 100. In an embodiment, the tube 100 may be formed from two panels. In such an embodiment, opposing edges of the two panels may be attached together, such as attached together using any of the techniques disclosed herein. The portions of the two films that are attached together may form features that preferentially fold. In an embodiment, the tube 100 may be formed from three or more panels that are attached together, for example, using any of the attachment techniques disclosed herein.

The one or more panels that form the tube 100 are formed from a fluid impermeable material which allows the one or more bodily fluids to flow within the conduit 108 while preventing the bodily fluids from leaking through the panels. The panels may also be formed of relatively flexible material(s) which allows the tube 100 to exhibit sharp bents therein without kinking. Examples of materials that may form the one or more panels includes polypropylene, polyethylene (e.g., low density polyethylene, linear low density polyethylene, medium density polyethylene, high density polyethylene, etc.), silicone, neoprene, rubber, flexible PVC, polyurethane, polyethylene terephthalate, a polycarbonate, a metal foil (e.g., aluminum foil), any other suitable fluid impermeable material, or combinations thereof. In an embodiment, the one or more panels are formed from an opaque (e.g., black) or frosted material to obscure visibility of the bodily fluids therein. In an embodiment, the tube 100 includes lay flat tubing.

In an embodiment, the one or more panels that form the tube 100 may exhibit a thickness. To keep the vertical profile of the tube 100 small as discussed above, the panels may exhibit a thickness that is about 0.03 mm or less, about 0.035 mm or less, about 0.04 mm or less, about 0.05 mm or less, about 0.06 mm or less, about 0.09 mm or less, about 0.12 mm or less, about 0.15 mm or less, about 0.18 mm or less, about 0.21 mm or less, about 0.24 mm or less, or in ranges of about 0.02 mm to about 0.03 mm, about 0.025 mm to about 0.035 mm, about 0.03 mm to about 0.4 mm, about 0.035 to about 0.05 mm, about 0.4 mm to about 0.06 mm, about 0.05 mm to about 0.09 mm, about 0.06 mm to about 0.12 mm, about 0.09 mm to about 0.15 mm, about 0.12 mm to about 0.18 mm, about 0.15 mm to about 0.21, or about 0.18 mm to about 0.24 mm. The thickness of the panels may generally be selected to be as small as possible to minimize the vertical profile of the tube 100. Further, the thickness of the panels may generally be selected to be as small as possible to maximize the flexibility of the tube 100. However, the thickness may need to be selected to withstand any forces that are applied thereto without ripping or otherwise failing. For example, a tube 100 that is subjected to a vacuum source and/or is configured to be disposed under the patient may need to relatively thicker (e.g., exhibit a thickness that is greater than 0.05 mm) to withstand the forces caused by the vacuum source and/or frictional wear. It is noted that, in some embodiments, the panels of the tube 100 may exhibit a thickness that is greater than 0.03 mm.

Referring to FIG. 1B, the tube 100 exhibits a width W and a thickness t that are measured perpendicular to a longitudinal axis 116 of the tube 100 and perpendicular to each other. The width W and the thickness t may refer to the maximum width and minimum thickness of the tube 100 after the tube 100 is positioned on a surface with no external forces (e.g., vacuum source, weight of a patient, etc.) applied thereto. In an embodiment, as shown, the width W and thickness t of the tube 100 is substantially uniform along the longitudinal axis 116 and, thus, the width W and thickness t of the tube 100 at the inlet 102, the outlet 104, and the intermediate portion 106 are the same. When there is no distinction between the widths and thicknesses of the inlet 102, the outlet 104, and the intermediate portion 106, the inlet 102 and the outlet 104 are merely the terminal ends of the tube 100. However, in other embodiments as will be discussed in more detail below, the width and thickness of at least one of the inlet 102 or the outlet 104 may be different than the width and thickness of the intermediate portion 106. In such embodiments, the inlet 102 and the outlet 104 are distinguishable from the intermediate portion 106 by the different widths and thicknesses thereof.

As previously discussed, the tube 100 exhibits an elongated cross-section because the width W is significantly greater than the thickness t. As used herein, the tube 100 exhibits an elongated cross-section and the width W is significantly greater than the thickness t when the width W is greater than the thickness t by about 5 times or more, about 7.5 times or more, about 10 times or more, about 12.5 times or more, about 15 times or more about 20 times or more about 25 times or more, about 30 times or more, about 40 times or more, about 50 times or more, about 75 times or more, about 100 times or more, about 150 times or more, about 200 times or more, or in ranges of about 5 to about 15 times, about 10 to about 20 times, about 15 to about 25 times, about 20 to about 30 times, about 25 to about 40 times, about 30 to about 50 times, about 40 to about 75 times, about 50 to about 100 times, about 75 to about 150 times, or about 100 to about 200 times. When the width W is significantly greater than the thickness t, the flexibility of the tube 100 is significantly increased relative to a conventional tube, especially when the tube 100 is bend along the width W. Further, when the width W is significantly greater than the thickness, the tube 100 may exhibit a vertical profile that is sufficiently small and/or distribute the weight of the patient along a large area of the tube 100 to allow the tube 100 to be positioned under a patient while minimizing the risk of bed sores caused by the tube 100.

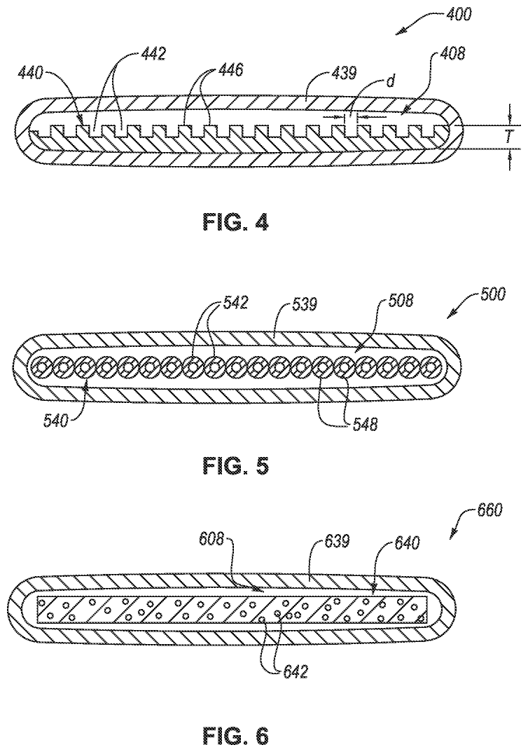

The width W of the tube 100 relative to the thickness t may be selected based on a number of factors. Generally, it is desirable to increase the width W of the tube 100 relative to the thickness t which may increase the flexibility of the tube 100, decrease the vertical profile of the tube 100, and reduce the likelihood that the tube 100 causes bed sores. However, increasing the width W relative to the thickness t may create several issues. For example, increasing the width W relative to the thickness t may increase the space required to store the tube 100 and require custom packaging (e.g., packaging that is distinct from the packaging that includes the fluid collection assembly). The width W and the thickness t may also be selected such that the cross-sectional area of the conduit 108 through which the bodily fluids may flow when at least one of no external forces are applied thereto, a vacuum is applied to the conduit 108, or the tube 100 is disposed under a patient is substantially the same as or similar to the cross-sectional area of a conduit of a conventional tube (e.g., about 30 mm² or greater). The cross-sectional area of the conduit 108 through which the bodily fluids may flow may also depend on whether the tube 100 includes one or more fluid flow features (see FIGS. 4-6) The width W and the thickness t may also be selected based on the hardness of the surface on which the tube 100 lies. For example, the width W relative to the thickness t may need to be increased when the tube 100 is on a hard surface to prevent bed sores than if the tube 100 is on a soft surface (e.g., a bed).

The thickness t of the tube 100 may affect the vertical profile. As such, the thickness t may be selected to be as small as possible to minimize the vertical profile to and minimize the likelihood that the tube 100 causes bed sores. For example, the thickness t may be selected to be about 0.01 mm or less, about 0.025 mm or less, about 0.05 mm or less, about 0.1 mm or less, about 0.25 mm or less, about 0.5 mm or less, about 0.75 mm or less, about 1 mm or less, about 1.25 mm or less, about 1.5 mm or less, about 1.75 mm or less, about 2 mm or less, about 2.5 mm or less, about 3 mm or less, about 3.5 mm or less, about 4 mm or less, or in ranges of about 0.01 mm to about 0.05 mm, about 0.025 mm to about 0.1 mm, about 0.05 mm to about 0.25 mm, about 0.1 mm to about 0.5 mm, about 0.25 mm to about 0.75 mm, about 0.5 mm to about 1 mm, about 0.75 mm to about 1.25 mm, about 1 mm to about 1.5 mm, about 1.25 mm to about 1.75 mm, about 1.5 mm to about 2 mm, about 1.75 mm to about 2.5 mm, about 2 mm to about 3 mm, about 2.5 mm to about 3.5 mm, or about 3 mm to about 4 mm. The thickness t of the tube 100 may depend on the thickness and flexibility of the one or more panels that form the tube 100. The thickness t of the tube 100 may also depend on whether the tube 100 includes one or more fluid flow features (illustrated in FIGS. 4-6).

The width W of the tube 100 may be about 1 mm or greater, about 2.5 mm or greater, about 5 mm or greater, about 7.5 mm or greater, about 10 mm or greater, about 12.5 mm or greater, about 15 mm or greater, about 17.5 mm or greater, about 20 mm or greater, about 25 mm or greater, about 30 mm or greater, about 40 mm or greater, about 50 mm or greater, about 60 mm or greater, about 80 mm or greater, about 100 mm or greater, or in ranges of about 1 mm to about 5 mm, about 2.5 mm to about 7.5 mm, about 5 mm to about 10 mm, about 7.5 mm to about 12.5 mm, about 10 mm to about 15 mm, about 12.5 mm to about 17.5 mm, about 15 mm to about 20 mm, about 17.5 mm to about 25 mm, about 20 mm to about 30 mm, about 25 mm to about 40 mm, about 30 mm to about 50 mm, about 40 mm to about 60 mm, about 50 mm to about 80 mm, or about 60 mm to about 100 mm. The width W of the tube 100 may be selected based on a number of factors. In an example, the width W of the tube 100 may be selected based on the thickness t of the tube 100 and the desired ratio of the width W to the thickness t. For instance, generally, increasing the width W of the tube 100 relative to the thickness t better distributes pressure across the tube 100 thereby reducing the likelihood that the tube 100 causes bed sores, even if the vertical profile of the tube 100 is greater than 1 mm, greater than 2 mm, or greater than 3 mm. The width W of the tube 100 may be also be selected based on whether the tube 100 includes a fluid flow feature. Generally, the fluid flow feature may increase the thickness t of the tube 100 thereby requiring increasing the width W of the tube 100 to be increased to accommodate the increased thickness t. Further, the fluid flow feature may decrease the cross-sectional area of the conduit 108 through which the bodily fluid may flow and the width W may be increased to ensure that the cross-sectional area of the conduit 108 remains large enough for the bodily fluids to flow therethrough.

The cross-sectional area of the conduit 108 through which the bodily fluids may flow depends on the width W and the thickness t of the tube 100. The cross-sectional area of the conduit 108 is generally selected such that the cross-sectional area, when no external forces are applied thereto and/or the tube 100 is disposed under a patient, is large enough that the bodily fluids present in the fluid collection assembly may be removed therefrom before that fluid collection assembly leaks. In an example, the cross-sectional area of the conduit 108 may be about 1 mm² or greater, about 5 mm² or greater, about 10 mm² or greater, about 15 mm² or greater, about 20 mm² or greater, 25 mm² or greater, about 30 mm² or greater, about 35 mm² or greater, about 40 mm² or greater, about 50 mm² or greater, about 75 mm² or greater, about 100 mm² or greater, or in ranges of about 1 mm² to 10 mm², 5 mm² to 15 mm², 10 mm² to 20 mm², 15 mm² to 25 mm², about 20 mm² to about 30 mm², about 25 mm² to about 35 mm², about 30 mm² to about 40 mm², about 35 mm² to about 50 mm², about 40 mm² to about 75 mm², or about 50 mm² to about 100 mm². In an example, the cross-sectional area of the conduit 108 may be selected to be equal to or greater than a cross-sectional area of an outlet of a fluid collection assembly or an inlet of a fluid storage container. In such an example, the cross-sectional area of the outlet of the fluid collection assembly or an inlet of a fluid storage container limits the amount of bodily fluids that flows through the conduit 108 and not the cross-sectional area of the conduit 108. In some instances, the cross-sectional area of the outlet of the fluid collection assembly or the inlet of the fluid storage container is about 30 mm² to about 35 mm².

As previously discussed, the tube 100 is illustrated with a break to indicate the tube 100 may have any suitable length. In an example, the tube 100 may exhibit a length that is about 30 cm or greater, about 60 cm or greater, about 1 m or greater, about 1.5 m or greater, about 2 m or greater, about 2.5 m or greater, about 3 m or greater, about 4 m or greater, about 5 m or greater or in ranges of about 30 cm to about 1 m, about 60 cm to about 1.5 m, about 1 m to about 2 m, about 1.5 m to about 2.5 m, about 2 m to about 3 m, about 2.5 m to about 4 m, or about 3 m to about 5 m.

As previously discussed, the inlet 102 and the outlet 104 have the same or substantially the same width W and thickness t as the intermediate portion 106. In an embodiment, the inlet 102 and/or the outlet 104 may have the same or substantially the same width W and thickness t as the intermediate portion 106 when tube 100 is integrally formed (e.g., exhibits single piece construction) with fluid collection assembly and/or fluid storage container, respectively. In an embodiment, the inlet 102 and/or the outlet 104 may have the same or substantially the same width W and thickness t as the intermediate portion 106 when the inlet 102 and/or outlet 104 are attached to one or more devices (e.g., fluid collection assembly, fluid storage container, or another tube) that is configured to be attached to (e.g., exhibit a width and thickness that is slightly larger or slightly smaller than) the inlet 102 and/or the outlet 104, respectively. In such an embodiment, the devices may be attached to the inlet 102 and/or outlet 104 via interference fit; stretching the inlet 102 and/or outlet 104 around the outlet or inlet of the devices, respectively; ultrasonically or radio frequency welding the devices to the inlet 102 and/or outlet 104; adhesively attaching the devices to the inlet 102 and/or outlet 104; or any other method of attaching the devices to the inlet 102 and/or outlet 104.

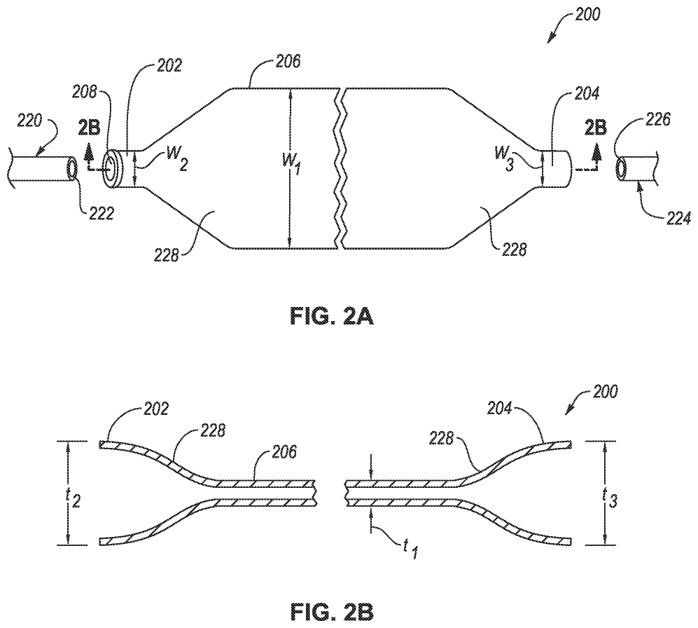

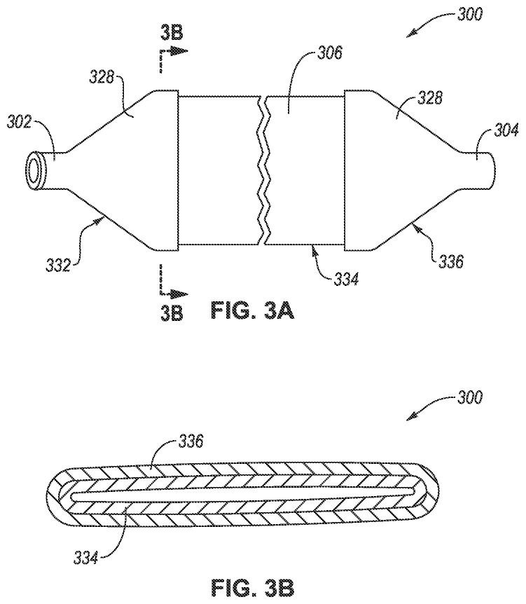

However, the tubes disclosed herein may be configured to be attached to one or more devices that are commonly available. For example, the tubes disclosed herein may be configured to be attached to one or more conventional fluid collection assemblies, one or more conventional fluid storage containers, one or more conventional tubes, or any other conventional device (hereinafter collectively referred to as “conventional devices”). The inlets and outlets of such conventional devices often exhibit a generally circular cross-sectional shape or other cross-sectional shape that does not exhibit a width that is significantly greater than the thickness thereof. As such, the tube 100 illustrated in FIGS. 1A and 1B may have difficulty being used with conventional devices. However, the tubes disclosed herein may include at least one of an inlet or an outlet that is configured to interface with (e.g., be directly attached to) one or more conventional devices. FIGS. 2A-3B illustrate examples of tubes that are configured to interface with one or more conventional devices, according to different embodiments.

FIG. 2A is an isometric view of a tube 200 with an elongated cross-section, according to an embodiment. FIG. 2B is a cross-sectional schematic of the tube 200 taken along plane 2B-2B shown in FIG. 2A, according to an embodiment. Except as otherwise disclosed herein, the tube 200 is the same or substantially similar to any of the tubes disclosed herein. For example, the tube 200 may include an inlet 202, an outlet 204, and an intermediate portion 206. The features of the tube 200 may be used in any of the embodiments disclosed herein.

The intermediate portion 206 of the tube 200 exhibits an intermediate width W₁ and an intermediate thickness t₁. The intermediate width W₁, the intermediate thickness t₁, and length of the intermediate portion 206 may be the same as or substantially similar to the width W and the thickness t of the tube 100 illustrated in FIGS. 1A and 1B. For instance, the intermediate width W₁ of the intermediate portion 206 may be significantly greater than the intermediate thickness t₁ thereof. In other words, at least the intermediate portion 206 exhibits the elongated cross-section of the tube 200

The inlet 202 may exhibit an inlet width W₂ and an inlet thickness t₂. The inlet 202 is distinguishable from the intermediate portion 206 because at least one of the inlet width W₂ or the inlet thickness t₂ of the inlet 202 is different (e.g., smaller or greater) than the intermediate width W₁ and the intermediate thickness t₁ of the intermediate portion 206, respectively. For example, the inlet width W₂ and the inlet thickness t₂ of the inlet 202 are selected to allow the inlet 202 to interface with a first conventional device 220 (illustrated as another tube). The first conventional device 220 includes an outlet 222 that exhibits a cross-sectional shape (e.g., a generally circular or any other suitable cross-sectional shape) and the inlet 202 exhibits a cross-sectional shape that corresponds to the generally circular cross-sectional shape of the outlet 222. In an example, the inlet 202 may be configured to receive the outlet 222 of the first conventional device 220 (e.g., the outlet 222 is positionable within the inlet 202). In such an example, the conduit 208 defined by the inlet 202 is slightly larger than the outlet 222 such that the outlet 222 may be positioned in the conduit 208. In an example, the outlet 222 of the first conventional device 220 is configured to receive the inlet 202 (e.g., the inlet 202 is positionable within the outlet 222). In such an example, the conduit defined by the first conventional device 220 is slightly larger than the inlet 202 such that the inlet 202 may be positioned within the conduit of the first conventional device 220. It is noted that, unlike the inlet 102 of FIGS. 1A and 1B, the inlet 202 may extend for a distance along the longitudinal axis of the tube 200.

The outlet 204 may exhibit an outlet width W₃ and an outlet thickness t₃. The outlet 204 is distinguishable from the intermediate portion 206 because at least one of the outlet width W₃ or the outlet thickness t₃ of the outlet 204 is different (e.g., smaller or greater) than the intermediate width W₁ and the intermediate thickness t₁ of the intermediate portion 206, respectively. For example, the outlet width W₃ and the outlet thickness t₃ of the outlet 204 are selected to allow the outlet 204 to interface with a second conventional device 224 (illustrated as another tube). The second conventional device 224 includes an inlet 226 that exhibits a cross-sectional shape (e.g., a generally circular or any other suitable cross-sectional shape) and the outlet 204 exhibits a cross-sectional shape that corresponds to the generally circular cross-sectional shape of the inlet 226. In an example, the outlet 204 may be configured to receive the inlet 226 of the second conventional device 224 (e.g., the inlet 226 is positionable within the outlet 204). In such an example, the conduit 208 defined by the outlet 204 is slightly larger than the inlet 226 such that the inlet 226 may be positioned in the conduit 208. In an example, the inlet 226 of the second conventional device 224 is configured to receive the outlet 204 (e.g., the outlet 204 is positionable within the inlet 226). In such an example, the conduit defined by the second conventional device 224 is slightly larger than the outlet 204 such that the outlet 204 may be positioned within the conduit of the second conventional device 224. It is noted that, unlike the outlet 104 of FIGS. 1A and 1B, the outlet 204 may extend for a distance along the longitudinal axis of the tube 200.

In an embodiment, the inlet width W₂ and the outlet width W₃ are less than the intermediate width W₁ and the inlet thickness t₂ and the outlet thickness t₃ are greater than the intermediate thickness t₁. For example, the intermediate width W₁ and the intermediate thickness t₁ are selected to be greater and smaller, respectively, than the widths and thicknesses of conventional devices thereby allowing the intermediate portion 206 to be more flexibility and decrease the likelihood that the intermediate portion 206 causes bed sores compared to conventional devices. Thus, the inlet width W₂ and the outlet width W₃ need to be smaller than the intermediate width W₁ of the intermediate portion 206 and the inlet thickness t₂ and the outlet thickness t₃ need to be larger than the intermediate thickness t₁ to interface with the conventional devices. It is noted that the intermediate portion 206 is generally the portion of the tube 200 that are selected to bend and contact the patient. As such, the decreased the inlet width W₂ and the outlet width W₃ relative to the intermediate width W₁ and the increased inlet thickness t₂ and the outlet thickness t₃ relative to the intermediate thickness t₁ does not adversely affect the function of the tube 200 (e.g., prevent bending of the tube 200 or increase the changes of bed sores). In some embodiments, at least one of the inlet width W₂, the outlet width W₃, the inlet thickness t₂, or the outlet thickness t₃ may be the same as the corresponding one of the intermediate width W₁ or intermediate thickness t₁.

The inlet width W₂ and the outlet width W₃ may be about 5 mm or less, about 6 mm or less, about 7 mm or less, about 8 mm or less, about 9 mm or less, about 10 mm or less, about 11 mm or less, about 12 mm or less, about 13 mm or less, about 15 mm or less, about 17.5 mm or less, about 20 mm or less, or in ranges of about 5 mm to about 7 mm, about 6 mm to about 8 mm, about 7 mm to about 9 mm, about 8 mm to about 10 mm, about 9 mm to about 11 mm, about 10 mm to about 12 mm, about 11 mm to about 13 mm, about 12 mm to about 15 mm, about 13 mm to about 17.5 mm, or about 15 mm to about 20 mm. In an example, the inlet width W₂ and the outlet width W₃ are selected based on the size (e.g., widths) of the outlet 222 and the inlet 226 of the first and second conventional devices 220, 224. In an example, the inlet width W₂ and the outlet width W₃ are selected based on whether the first and second conventional devices 220, 224 are configured to receive or be positioned within respective ones of the inlet 202 or the outlet 204. In an embodiment, the inlet width W₂ and the outlet width W₃ are the same or substantially the same, such as when the first and second conventional devices 220, 224 exhibit substantially the same width. In an embodiment, the inlet width W₂ and the outlet width W₃ are different, such as when the first and second conventional devices 220, 224 exhibit different widths.

The inlet thickness t₂ and the outlet thickness t₃ may be about 1 mm or greater, about 3 mm or greater, about 4 mm or greater, about 5 mm or less, about 6 mm or less, about 7 mm or less, about 8 mm or less, about 9 mm or less, about 10 mm or less, about 11 mm or less, about 12 mm or less, about 13 mm or less, about 15 mm or less, about 17.5 mm or less, about 20 mm or less, or in ranges of about 1 mm to about 5 mm, about 3 mm to about 6 mm, about 5 mm to about 7 mm, about 6 mm to about 8 mm, about 7 mm to about 9 mm, about 8 mm to about 10 mm, about 9 mm to about 11 mm, about 10 mm to about 12 mm, about 11 mm to about 13 mm, about 12 mm to about 15 mm, about 13 mm to about 17.5 mm, or about 15 mm to about 20 mm. In an example, the inlet thickness t₂ and the outlet thickness t₃ are selected based on the size (e.g., thickness) of the outlet 222 and the inlet 226 of the first and second conventional devices 220, 224. In an example, the inlet thickness t₂ and the outlet thickness t₃ are selected based on whether the first and second conventional devices 220, 224 are configured to receive or be positioned within respective ones of the inlet 202 or the outlet 204. In an embodiment, the inlet thickness t₂ and the outlet thickness t₃ are the same or substantially the same, such as when the first and second conventional devices 220, 224 exhibit substantially the same thickness. In an embodiment, the inlet thickness t₂ and the outlet thickness t₃ are different, such as when the first and second conventional devices 220, 224 exhibit different thicknesses.

In an embodiment, at least one of the inlet width W₁ or the outlet width W₂ may be smaller than, substantially equal to, or at most about 4 times greater (e.g., at most about 1.5 times greater, at most about 2 times greater, at most about 3 times greater, or in ranges of about 1 to about 2 times greater, about 1.5 to about 3 times greater, or about 2 to about 4 times greater) than the inlet thickness t₂ or the outlet thickness t₃, respectively. In such an embodiment, the size of the inlet width W₁ relative to the inlet thickness t₂ and/or the size of the outlet width W₃ relative to the outlet thickness t₃ may facilitate attachment of the inlet 202 and/or the outlet 204 to a convention tube since conventional tubes exhibit similar widths relative to the thickness thereof. In other words, the inlet 202 and the outlet 204 may not exhibit an elongated cross-section.

In an embodiment, the tube 200 includes two tapered portions 228. One of the two tapered portions 228 is positioned and extending between the intermediate portion 206 and the inlet 202 while the other one of the two tapered portions 228 is positioned and extends between the intermediate portion 206 and the outlet 204. The tapered portions 228 exhibits a width and a thickness measured perpendicularly to the longitudinal axis 216 of the tube 200. At least one of the widths or thicknesses of the tapered portions 228 vary along the longitudinal axis 216. For example, the widths of the tapered portion 228 may vary (e.g., increase) from the inlet width W₂ and the outlet width W₃ of the corresponding one of the inlet 202 and the outlet 204 to the intermediate width W₁ of the intermediate portion 206. The thickness of the tapered portions 228 may vary (e.g., decrease) from the inlet thickness t₂ and the outlet thickness t₃ of the corresponding one of the inlet 202 and the outlet 204 to the intermediate thickness t₁ of the intermediate portion 206. In other words, the tapered portions 228 allows the widths and thicknesses of the tube 200 to gradually increase and/or decrease between the intermediate portion 206 and the corresponding one of the inlet 202 and the outlet 204. Generally, increasing or decreasing the widths and thickness of the tube 200 may facilitate fluid flow therein by minimizing the likelihood that the bodily fluids stagnate in the tube 200. In an embodiment, one or more of the tapered portions 228 are omitted from the tube 200 and the widths and/or thicknesses of the tube 200 suddenly changes where the intermediate portion 206 meets one or more of the inlet 202 or the outlet 204. In such an embodiment, the bodily fluids flowing through the conduit 208 may be more likely to pool where the widths and or thickness of the tube 200 suddenly change than if the tube 200 includes the tapered portions 228.

In an embodiment, at least one of the inlet width W₂, the outlet width W₃, the inlet thickness t₂, or the outlet thickness t₃ is the same or substantially similar to a corresponding one the intermediate width W₁ and the intermediate thickness t₁ of the intermediate portion 206.

The inlet 202, the outlet 204, the intermediate portion 206, and the tapered portions 228 of the tube 200 are integrally formed together (e.g., exhibits single piece construction). In other words, there are no seams between adjacent ones of the inlet 202, the outlet 204, the intermediate portion 206, and the tapered portions 228. However, at least one of the inlet, the outlet, the intermediate portion, or the tapered portions of any of the tubes disclosed herein may be distinct from and attached to the other ones of the inlet, the outlet, the intermediate portion, or the tapered portions. For example, FIG. 3A is an isometric view of a tube 300 with an elongated cross-section that is formed from a plurality of distinct components that are attached together, according to an embodiment. FIG. 3B is a cross-sectional schematic of the tube 300 taken along plane 3B-3B, according to an embodiment. Except as otherwise disclosed herein, the tube 300 is the same or substantially similar to any of the tubes disclosed herein. For example, the tube 300 may include an inlet 302, and outlet 304, an intermediate portion 306, and two tapered portions 328. Further, the features of the tube 300 may be used in any of the embodiments disclosed herein.

The tube 300 is formed from two or more distinct portions that are attached to each other. For example, in the illustrated embodiment, the tube 300 includes a first piece 332, a second piece 334, and a third piece 336. It is noted that the tube 300 may only include two portions or four or more portions, without limitation. The first piece 332 may include the inlet 302, the second piece 334 may include at least a portion of the intermediate portion 306, and the third piece 336 may include the outlet 304. In the illustrated embodiment, the first and third pieces 332, 336 also include one of the tapered portions 338 and a portion of the intermediate portion 306. However, it is noted that the second piece 334 may include at least one of at least a portion of the tapered portions 328 instead of the first and/or second pieces 332, 334 or at least one of the tapered portions 328 may be omitted.

The first piece 332 may be attached to one end of the second piece 334 and the third piece 336 may be attached to the opposing end of the second piece 334. For example, a portion of the second piece 334 may be disposed within a portion of the conduit 308 defined by the first piece 332 or vice versa and an opposing portion of the second piece 334 may be disposed within a portion of the conduit 308 defined by the third piece 336 or vice versa. The overlapping portions of the first piece 332 and the second piece 334 and the overlapping portions of the second piece 334 and the third piece 336 may be attached together using any suitable attachment technique. For example, the overlapping portions of the first, second, and third pieces 332, 334, 336 may be attached together using an adhesive, ultrasonic welding, or radio frequency welding.

Forming the tube 300 from a plurality of distinct portions may facilitate attaching the tube 300 to the conventional devices previously discussed herein or any other device. For example, the second piece 334 may be formed from a relatively flexible and/or relatively thin material. The relatively flexible and/or relatively thin material allows the second piece 334 to be easily bent and to be positioned under the patient without causing bed sores. However, the relatively flexible and/or relatively thin material may be difficult to attach to other devices, for example, using an interference fit. The first and second pieces 332, 336 may be formed from a material that is more rigid and/or thicker than the second piece 334 which may facilitate attaching the inlet 302 and the outlet 304 to other devices, for example, with an interference fit. The first and third pieces 332, 336 are likely to be positioned adjacent to the other devices of a fluid collection system and, thus, are unlikely to be bent or positioned under a patient. As such, the increased rigidity and/or thickness of the first and third pieces 332, 336 relative to the second piece 334 are unlikely to adversely affect the function of the tube 300.

In an embodiment, forming the tube 300 may facilitate formation of the tube 300. In an example, at least one of the first, second, or third pieces 332, 334, 336 may be formed using a process that is different than the other one(s) of the first, second, or third pieces 332, 334, 336. The different processes used to form the first, second, and third pieces 332, 334, 336 may allow the different pieces to be formed using more efficient processes. For instance, the second piece 334 may be formed using an extrusion process, such as a blown film extrusion process, or a tape casting process. Such extrusion and/or tape casting processes may allow the second piece 334 to be formed having relatively long lengths and relatively thin panels than if the second piece 334 was formed, for example, using an injection molding process. The first and third pieces 332, 336, however, may be formed using an injection molding process. In an example, forming the tube 300 from a plurality of pieces may allow one or more fluid flow features (shown in FIGS. 4-6) that are too large to fit through the inlet 302 or the outlet 304 to be inserted into the conduit 308 before at least two of the first, second, or third pieces 332, 334, 336 are attached together.

Any of the tubes disclosed herein may include one or more fluid flow features disclosed in the conduits thereof. The fluid flow features define a plurality of channels therein that are configured to facilitate flow of the bodily fluids therethrough. For example, the fluid flow features may prevent or at least inhibit the collapse of the channels when the tube is disposed under a patient which would otherwise prevent the bodily fluids from flowing through the tubes. FIGS. 4-6 are cross-sectional schematics of different tubes with elongated cross-sections that include different fluid flow features, according to different embodiments.

Referring to FIG. 4, the tube 400 includes one or more panels 439 that define a conduit 408. The tube 400 also include at least one fluid flow feature 440 disposed in the conduit 408. The fluid flow feature 440 partially occupies the conduit 408. The fluid flow feature 440 defines a plurality of channels 442 through which the bodily fluids may flow. For example, the fluid flow feature 440 includes at least one base 444 and a plurality of walls 446 extending from the base 444. The base 444 and the walls 446 form a plurality of U-shaped that define the channels 442. The base 444 and the walls 446 may be formed from any of the materials disclosed herein. In an example, at least a portion of at least one of the base 444 or the walls 446 may be formed from any of the fluid impermeable materials disclosed herein. In an example, at least a portion of at least one of the base 444 or the walls 446 may be formed from a metal, such as aluminum, stainless steel, or copper. In such an example, the metal may be coated with a polymer to prevent the metal from puncturing the panels of the tube 400.

The fluid flow feature 440 may exhibit a thickness T. The thickness T may be selected to be 0.25 mm or less, about 0.5 mm or less, about 0.75 mm or less, about 1 mm or less, about 1.25 mm or less, about 1.5 mm or less, about 1.75 mm or less, about 2 mm or less, about 2.5 mm or less, about 3 mm or less, or in ranges of about 0.25 mm to about 0.75 mm, about 0.5 mm to about 1 mm, about 0.75 mm to about 1.25 mm, about 1 mm to about 1.5 mm, about 1.25 mm to about 1.75 mm, about 1.5 mm to about 2 mm, about 1.75 mm to about 2.5 mm, or about 2 mm to about 3 mm. In an example, the thickness T of the fluid flow feature 440 may be selected based on the desired overall thickness t of the tube 400. In an example, the thickness T of the fluid flow feature 440 may be selected such that the cross-sectional area of each of the channels 442 is greater than the cross-sectional area of an outlet of a fluid collection assembly or the inlet of a fluid storage container. In such an example, the cross-sectional area of the outlet of the fluid collection assembly or the inlet of the fluid storage container controls the amount of bodily fluids flowing through the conduit 408 and not the cross-sectional area of the channels 442.

The walls 446 may be spaced from each other by a distance d. The distance d may be selected to be 0.25 mm or less, about 0.5 mm or less, about 0.75 mm or less, about 1 mm or less, about 1.25 mm or less, about 1.5 mm or less, about 1.75 mm or less, about 2 mm or less, about 2.5 mm or less, about 3 mm or less, or in ranges of about 0.25 mm to about 0.75 mm, about 0.5 mm to about 1 mm, about 0.75 mm to about 1.25 mm, about 1 mm to about 1.5 mm, about 1.25 mm to about 1.75 mm, about 1.5 mm to about 2 mm, about 1.75 mm to about 2.5 mm, or about 2 mm to about 3 mm. In an example, the distance d of the fluid flow feature 440 may be selected such that the cross-sectional area of each of the channels 442 is greater than the cross-sectional area of an outlet of a fluid collection assembly or the inlet of a fluid storage container. In such an example, the cross-sectional area of the outlet of the fluid collection assembly or the inlet of the fluid storage container controls the amount of bodily fluids flowing through the conduit 408 and not the cross-sectional area of the channels 442. In an example, the distance d is selected such that the fluid flow feature 440 may support the weight of a portion of a patient under which the tube 400 is positioned while preventing collapse of most (e.g., at least 50%) of the channels 442. For instance, increasing the distance d decreases the number of walls 446 that may support the portion of the patient for a given thickness of the walls 446. Decreasing the number of walls 446 that may support the portion of the patient also decreases the maximum weight that the fluid flow feature 440 may support before the channels 442 begin to collapse. Thus, the distance d may be selected based on balancing the cross-sectional area of the channels 442 while preventing the collapse of the channels 442.

Referring to FIG. 5, the tube 500 includes one or more panels 539 that define a conduit 508. The tube 500 also include at least one fluid flow feature 540 disposed in the conduit 508. The fluid flow feature 540 partially occupies the conduit 508. Except as otherwise disclosed herein, the fluid flow feature 540 may exhibit any of the characteristics of the fluid flow feature 540 of FIG. 5. For example, the fluid flow feature 540 may exhibit any of the same thicknesses as the fluid flow feature 540 and the channels 542 defined by the fluid flow feature 540 may have any of the same cross-sectional areas as the channels 442 of the fluid flow feature 440.

The fluid flow feature 540 includes a plurality of pipes 548 (e.g., micro-tubing). Each of the plurality of pipes 548 are hollow and define a channel 542. The plurality of pipes 548 may be aligned in one or more rows. Preferably, the pipes 548 are aligned in a single row (as shown) to minimize the overall thickness of the fluid flow feature 540. However, the plurality of pipes 548 may be aligned in a plurality of rows, for example, the increase the cross-sectional area of the channels 542. In an example, when the plurality of pipes 548 are aligned in a plurality of rows, adjacent rows of pipes 548 may be offset relative to each other. In such an example, the pipes 548 in one row are partially positioned in the groove between adjacent pipes 548 of another row such that the plurality of pipes 548 exhibit a more densely packed configuration (e.g., a close packed configuration) then if the adjacent rows of pipes 548 are not offset.

Referring to FIG. 6, the tube 600 includes one or more panels 639 that define a conduit 608. The tube 600 includes a fluid flow feature 640 disposed in the conduit 608. Except as otherwise disclosed herein, the fluid flow feature 640 may exhibit any of the characteristics of any of the fluid flow features disclosed herein.

The fluid flow feature 640 is a porous structure (e.g., a sponge-like structure). The porous structure defines a plurality of pores 642 that form the channels of the fluid flow feature 640. The plurality of pores 642 may be interconnected such that the bodily fluids may flow through the fluid flow feature 640 even when one or more of the pores 642 collapses. The porous structure may include a polymer foam, a metal foam, fabric (e.g., cotton, nylon, etc.), a wire mesh structure, or any other porous material.

In an embodiment, the fluid flow features of any of the tubes disclosed herein may exhibit a structure other than the structures illustrated in FIGS. 4-6. For example, the fluid flow features may include recesses formed in, one or more ridges that extend outwardly from, or walls extending between adjacent portions of the one or more panels that form the tube. In an example, the fluid flow features of any of the tubes disclosed herein may include a combination of any of the fluid flow features disclosed herein.

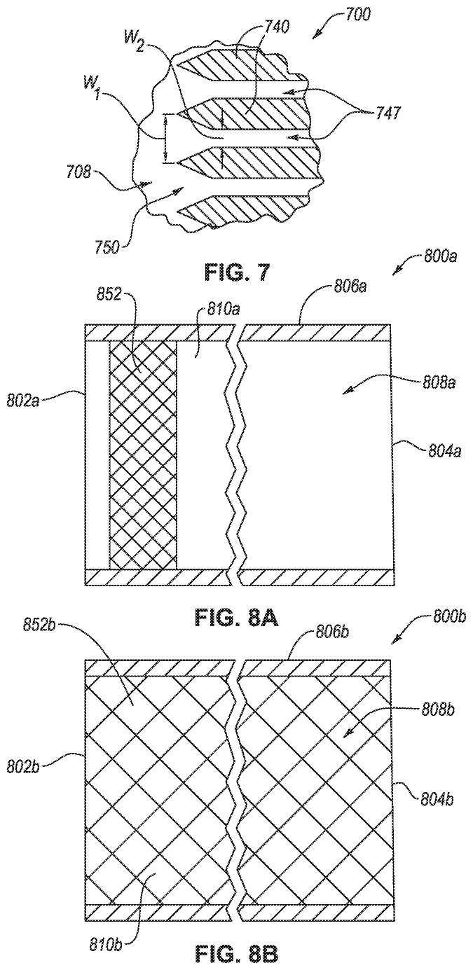

FIG. 7 is a cross-sectional schematic of a portion of a tube 700, according to an embodiment. Wherein the tubes 400, 500, 600 are cross-sectional schematics of the tubes taken along a plane that is parallel to the thickness and widths of such tubes, the cross-sectional schematic of the tube 700 is taken along a plane that is parallel to the longitudinal axis and width of the tube 700. Except as otherwise disclosed herein, the tube 700 may be the same or substantially similar to any of the tubes disclosed herein. Further, the features of the tube 700 may be used in any of the embodiments disclosed herein.

The tube 700 defines a conduit 708. The tube 700 includes a fluid flow features 740 disposed in the conduit 708. The fluid flow feature 740 may include any of the fluid flow features 740 disclosed herein. The fluid flow feature 740 defines a plurality of channels 742. Each of the channels 742 includes an entrance 750 and an exit (not shown) positioned downstream from the entrance 750. In other words, the entrance 750 of the channels 742 is positioned closed to the inlet (not show) of the tube 700 than the exit while the exit is positioned closer to the outlet (not shown) of the tube 700 than the entrance 750.

The channels 742 are configured to encourage flow of the bodily fluids from the entrance 750 to the exit, thereby inhibiting backflow of the bodily fluids. Backflow of the bodily fluids may cause leaks and unsanitary conditions. In an embodiment, the widths of the channels 742 are configured to encourage flow of the bodily fluids from the entrance 750 to the exit. In such an embodiment, the channels 742 may include a first width W₁ at the entrance 750. The width of the channels 742 may decrease with increasing distance from the entrance 750 until the channels 742 exhibit a second width W₂ that is less than the first width W₁. Decreasing the widths of the channels 742 increases the forces applied to the bodily fluids via capillary action. In other words, the forces applied to the bodily fluids via capillary action at the entrance 750 is less than the forces applied to the bodily fluids via capillary action at the portion of the channel 742 that exhibits the second width W₂. Thus, the decreasing width of the channels 742 encourages the bodily fluids to flow from the entrance 750 towards the exit.

It is noted that the width of the channels 742 do not have to start decreasing at the entrance 750. Instead, the widths of the channels 742 may remain substantially constant from the entrance 750 for a distance. After the distance, the width of the channels 742 may decrease from the first width W₁ to the second width W₂. The pressure differential caused by encouraging the bodily fluids to flow from the first width W₁ to the second width W₂ may pull the bodily fluids downstream from the entrance 750. In an embodiment, the location at which the widths of the channels 742 decrease may vary from channel 742 to channel 742. In an embodiment, only some of the channels 742 may exhibit a width that decreases while the remaining channels 742 do not.

FIG. 8A is a cross-sectional schematic of a tube 800a having an elongated cross-section, according to an embodiment. Except as otherwise disclosed herein, the tube 800a is the same or substantially similar to any of the tubes disclosed herein. Further, the features of the tube 800a may be used in any of the embodiments disclosed herein.

The tube 800a includes an inlet 802a, an outlet 804a, and an intermediate portion 806a. The tube 800a also defines an conduit 808a. A portion of an inner surface 810a of the tube 800a includes a disinfectant coating 852a (shown schematically with cross-hatching) disposed thereon. In an embodiment, the disinfectant coating 852a may be disposed on a portion of the inner surface 810a that is proximate to the inlet 802a (e.g., between a fluid flow feature, not shown, and the inlet 802a). As such, the disinfectant coating 852a may be positioned to disinfect bodily fluids that backflow towards the inlet 802a thereby minimizing unsanitary conditions caused by the backflow. The disinfectant coating 852a may also be disposed on at least a portion of the surfaces of a fluid flow feature (not shown) that define the channels of the fluid flow feature at and/or near the entrances thereof.

The disinfectant coating 852a may include any material that may at least one of kill bacteria, kill viruses, kill mold or fungus, kill other microorganisms, or react with chemicals that are present in the bodily fluids. For example, the disinfectant coating 852a may include at least one of a silver, silver ions, copper, copper ions, other disinfectants, or combinations thereof.

The disinfectant coating 852a may be disposed on portions of the inner surface 810a other than portions that are proximate to the inlet 802a, without limitation. For example, FIG. 8B is a cross-sectional schematic of a tube 800b having an elongated cross-section, according to an embodiment. Except as otherwise disclosed herein, the tube 800b is the same or substantially similar to any of the tubes disclosed herein. Further, the features of the tube 800b may be used in any of the embodiments disclosed herein. The tube 800b includes an inlet 802b, and outlet 804b, and an intermediate portion 806b. The tube 800b include at least one inner surface 810b that defines the conduit 808b. Substantially all of the inner surface 810b includes a disinfecting coating 852b disposed therein.

It is noted that the tubes 800a, 800b are illustrated as being substantially similar to the tube 100 of FIGS. 1A and 1B. However, it is noted that the tubes 800a, 800b may be the same or substantially similar to any of the other tubes disclosed herein. For example, the tubes 800a, 800b may include one or more tapered portions or a fluid flow feature.

As previously discussed, the tubes disclosed herein may be connected, either directly or indirectly, to one or more fluid collection assemblies. In an embodiment, the fluid collection assembly to which any of the tubes are connected may include a female fluid collection assembly configured to collect bodily fluids from a female patient. FIG. 9A is an isometric view of fluid collection system 951 that includes a female fluid collection assembly 952 connected to a tube 900, according to an embodiment. FIGS. 9B and 9C are cross-sectional schematics of the female fluid collection system 951 taken along planes 9B-9B and 9C-9C as shown in FIG. 9A, according to an embodiment. The fluid collection assembly 952 includes a fluid impermeable barrier 954, porous material 956 disposed in a chamber 958 within the fluid impermeable barrier 954, and an optional assembly tube 964 at least partially disposed within the chamber 958.

The fluid impermeable barrier 954 at least partially defines a chamber 958 (e.g., interior region) and an opening 960. For example, the interior surface(s) 961 of the fluid impermeable barrier 954 at least partially defines the chamber 958 within the fluid collection assembly 952. The fluid impermeable barrier 954 temporarily stores the bodily fluids in the chamber 958. The fluid impermeable barrier 954 may be formed of any suitable fluid impermeable material(s), such as a fluid impermeable polymer (e.g., silicone, polypropylene, polyethylene, polyethylene terephthalate, a polycarbonate, etc.), a metal film, natural rubber, another suitable material, or combinations thereof. As such, the fluid impermeable barrier 954 substantially prevents the bodily fluids from passing through the fluid impermeable barrier 954. In an example, the fluid impermeable barrier 954 may be air permeable and fluid impermeable. In such an example, the fluid impermeable barrier 954 may be formed of a hydrophobic material that defines a plurality of pores. At least one or more portions of at least an outer surface 962 of the fluid impermeable barrier 954 may be formed from a soft and/or smooth material, thereby reducing chaffing.

In some examples, the fluid impermeable barrier 954 may be tubular (ignoring the opening), such as substantially cylindrical (as shown), oblong, prismatic, or flattened. During use, the outer surface 962 of the fluid impermeable barrier 954 may contact the patient. The fluid impermeable barrier 954 may be sized and shaped to fit in the gluteal cleft between the legs of a female user.

The opening 960 provides an ingress route for fluids to enter the chamber 958. The opening 960 may be defined by the fluid impermeable barrier 954 such as by an inner edge of the fluid impermeable barrier 954. For example, the opening 960 is formed in and extends through the fluid impermeable barrier 954, from the outer surface 962 to the inner surface 961, thereby enabling bodily fluids to enter the chamber 958 from outside of the fluid collection assembly 952. The opening 960 may be an elongated hole in the fluid impermeable barrier 954. For example, the opening 960 may be defined as a cut-out in the fluid impermeable barrier 954. The opening 960 may be located and shaped to be positioned adjacent to a female urethra.

The fluid collection assembly 952 may be positioned proximate to the female urethral opening and the bodily fluids may enter the chamber 958 of the fluid collection assembly 952 via the opening 960. The fluid collection assembly 952 is configured to receive the bodily fluids into the chamber 958 via the opening 960. When in use, the opening 960 may have an elongated shape that extends from a first location below the urethral opening (e.g., at or near the anus or the vaginal opening) to a second location above the urethral opening (e.g., at or near the top of the vaginal opening or the pubic hair).

The opening 960 may have an elongated shape because the space between the legs of a female is relatively small when the legs of the female are closed, thereby only permitting the flow of the bodily fluids along a path that corresponds to the elongated shape of the opening 960 (e.g., longitudinally extending opening). The opening 960 in the fluid impermeable barrier 954 may exhibit a length that is measured along the longitudinal axis of the fluid collection assembly 952 that may be at least about 10% of the length of the fluid collection assembly 952, such as about 25% to about 50%, about 40% to about 50%, about 50% to about 75%, about 55% to about 85%, or about 75% to about 95% of the length of the fluid collection assembly 952.

The opening 960 in the fluid impermeable barrier 954 may exhibit a width that is measured transverse to the longitudinal axis of the fluid collection assembly 952 that may be at least about 10% of the circumference of the fluid collection assembly 952, such as about 25% to about 50%, about 40% to about 50%, about 50% to about 75%, about 55% to about 85%, or about 75% to about 90% of the circumference of the fluid collection assembly 952. The opening 960 may exhibit a width that is greater than 50% of the circumference of the fluid collection assembly 952 since the vacuum (e.g., suction) through the assembly tube 964 pulls the fluid through the porous material 956 and into the assembly tube 964. In some examples, the opening 960 may be vertically oriented (e.g., having a major axis parallel to the longitudinal axis of the fluid collection assembly 952). In some examples (not shown), the opening 960 may be horizontally oriented (e.g., having a major axis perpendicular to the longitudinal axis of the fluid collection assembly 952). In an example, the fluid impermeable barrier 954 may be configured to be attached to the patient, such as adhesively attached (e.g., with a hydrogel adhesive) to the patient. According to an example, a suitable adhesive is a hydrogel layer.

In some examples, the fluid impermeable barrier 954 may define an fluid outlet 965 sized to receive the assembly tube 964. The at least one assembly tube 964 may be disposed in the chamber 958 via the fluid outlet 965. The fluid outlet 965 may be sized and shaped to form an at least substantially fluid tight seal against the assembly tube 964 or the at least one tube thereby substantially preventing the bodily fluids from escaping the chamber 958.

The fluid impermeable barrier 954 may include markings thereon (not shown), such as one or more markings to aid a user in aligning the fluid collection assembly 952 on the patient. For example, a line on the fluid impermeable barrier 954 (e.g., opposite the opening 960) may allow a healthcare professional to align the opening 960 over the urethra of the patient. In examples, the markings may include one or more of alignment guide or an orientation indicator, such as a stripe or hashes. Such markings may be positioned to align the fluid collection assembly 952 to one or more anatomical features such as a pubic bone, etc.

The fluid collection assembly 952 includes porous material 956 disposed in the chamber 958. The porous material 956 may cover at least a portion (e.g., all) of the opening 960. The porous material 956 is exposed to the environment outside of the chamber 958 through the opening 960. In an embodiment, the porous material 956 may be configured to wick any bodily fluids away from the opening 960, thereby preventing the bodily fluids from escaping the chamber 958. The permeable properties referred to herein may be wicking, capillary action, diffusion, or other similar properties or processes, and are referred to herein as “permeable” and/or “wicking.” Such “wicking” may not include absorption of the bodily fluids into the wicking material. Put another way, substantially no absorption of the bodily fluids into the material may take place after the material is exposed to the bodily fluids and removed from the bodily fluids for a time. While no absorption is desired, the term “substantially no absorption” may allow for nominal amounts of absorption of the bodily fluids into the wicking material (e.g., absorbency), such as less than about 10 wt % of the dry weight of the wicking material, less than about 7 wt %, less than about 5 wt %, less than about 3 wt %, less than about 2 wt %, less than about 1 wt %, or less than about 0.5 wt % of the dry weight of the wicking material. The wicking material 956 may also wick the bodily fluids generally towards an interior of the chamber 958, as discussed in more detail below. In an embodiment, the porous material 956 may include at least one absorbent or adsorbent material.

The porous material 956 may include the fluid permeable membrane 966 disposed in the chamber 958. The fluid permeable membrane 966 may cover at least a portion (e.g., all) of the opening 960. The fluid permeable membrane 966 may be composed to wick the bodily fluids away from the opening 960, thereby preventing the bodily fluids from escaping the chamber 958.

In an embodiment, the fluid permeable membrane 966 may include any material that may wick the bodily fluids. For example, the fluid permeable membrane 966 may include fabric, such as a gauze (e.g., a silk, linen, or cotton gauze), another soft fabric, or another smooth fabric. Forming the fluid permeable membrane 966 from gauze, soft fabric, and/or smooth fabric may reduce chaffing caused by the fluid collection assembly 952.

The porous material 956 may include the fluid permeable support 968 disposed in the chamber 958. The fluid permeable support 968 is configured to support the fluid permeable membrane 966 since the fluid permeable membrane 966 may be formed from a relatively foldable, flimsy, or otherwise easily deformable material. For example, the fluid permeable support 968 may be positioned such that the fluid permeable membrane 966 is disposed between the fluid permeable support 968 and the fluid impermeable barrier 954. As such, the fluid permeable support 968 may support and maintain the position of the fluid permeable membrane 966. The fluid permeable support 968 may include any material that may wick the bodily fluids, such as any of the fluid permeable membrane materials disclosed herein above. For example, the fluid permeable membrane material(s) may be utilized in a more dense or rigid form than in the fluid permeable membrane 966 when used as the fluid permeable support 968. The fluid permeable support 968 may be formed from any fluid permeable material that is less deformable than the fluid permeable membrane 966. For example, the fluid permeable support 968 may include a porous polymer (e.g., nylon, spun nylon fibers, polyester, polyurethane, polyethylene, polypropylene, etc.) structure or an open cell foam. In some examples, the fluid permeable support 968 may be formed from a natural material, such as cotton, wool, silk, or combinations thereof. In such examples, the material may have a coating to prevent or limit absorption of fluid into the material, such as a water repellent coating. In some examples, the fluid permeable support 968 may be formed from fabric, felt, gauze, or combinations thereof.

In some examples, the fluid permeable membrane 966 may be optional. For example, the porous material 956 may include only the fluid permeable support 968. In some examples, the fluid permeable support 968 may be optionally omitted from the fluid collection assembly 952. For example, the porous material 956 may only include the fluid permeable membrane 966.

The fluid permeable support 968 may have a greater ability to wick the bodily fluids than the fluid permeable membrane 966, such as to move the bodily fluids inwardly from the outer surface 962 of the fluid collection assembly 952. In some examples, the porous ability of the fluid permeable support 968 and the fluid permeable membrane 966 may be substantially the same.

The fluid permeable membrane 966 and the fluid permeable support 968 may at least substantially completely fill the portions of the chamber 958 that are not occupied by the assembly tube 964. In some examples, the fluid permeable membrane 966 and the fluid permeable support 968 may not substantially completely fill the portions of the chamber 958 that are not occupied by the assembly tube 964. In such an example, the fluid collection assembly 952 includes the reservoir 969 (FIG. 9C) disposed in the chamber 958.

The reservoir 969 is a substantially unoccupied portion of the chamber 958. The reservoir 969 may be defined between the fluid impermeable barrier 954 and one or both of the fluid permeable membrane 966 and fluid permeable support 968. The bodily fluids that are in the chamber 958 may flow through the fluid permeable membrane 966 and/or fluid permeable support 968 to the reservoir 969. The reservoir 969 may retain of the bodily fluids therein.

The bodily fluids that are in the chamber 958 may flow through the fluid permeable membrane 966 and/or fluid permeable support 968 to the reservoir 969. The fluid impermeable barrier 954 may retain the bodily fluids in the reservoir 969. While depicted in the second end region 972, the reservoir 969 may be located in any portion of the chamber 958 such as the first end region 970. The reservoir 969 may be located in a portion of the chamber 958 that is designed to be located in a gravimetrically low point of the fluid collection assembly 952 when the fluid collection assembly 952 is worn.

In some examples (not shown), the fluid collection assembly 952 may include multiple reservoirs, such as a first reservoir that is located at the portion of the chamber 958 closest to the inlet of the assembly tube 964 (e.g., second end region 972) and a second reservoir that is located at the portion of the of the chamber 958 that is closest to the outlet of the assembly tube 964 (e.g., first end region 970). In another example, the fluid permeable support 968 is spaced from at least a portion of the tube 964, and the reservoir 969 may be the space between the fluid permeable support 968 and the assembly tube 964.

The assembly tube 964 may be at least partially disposed in the chamber 958. The assembly tube 964 may be used to remove the bodily fluids from the chamber 958. The assembly tube 964 includes an inlet of the assembly tube 964 and an outlet of the assembly tube 964 positioned downstream from the inlet of the assembly tube 964. The outlet of the assembly tube 964 may be operably coupled to a suction source, such as a vacuum pump for withdrawing fluid form the chamber 958 through the assembly tube 964. For example, the assembly tube 964 may extend into the fluid impermeable barrier 954 from the first end region 970 and may extend to the second end region 972 to a point proximate to the reservoir 969 therein such that the inlet of the assembly tube 964 is in fluid communication with the reservoir 969. The assembly tube 964 fluidly couples the chamber 958 with the fluid storage container (not shown) or the vacuum source (not shown).

The assembly tube 964 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the assembly tube 964 may include silicon or latex. In some examples, the assembly tube 964 may include one or more portions that are resilient, such as to by having one or more of a diameter or wall thickness that allows the assembly tube 964 to be flexible.

The assembly tube 964 may extend through a bore in the fluid permeable membrane 966 and/or fluid permeable support 968, such as into the reservoir 969. For example, the inlet of the assembly tube 964 may be extend into or be positioned in the reservoir 969. In the illustrated embodiment, the assembly tube 964 is at least partially disposed in the reservoir 969. In some examples (not shown), the assembly tube 964 may enter the chamber 958 in the second end region 970 and the inlet of the assembly tube 964 of the assembly tube 964 may be disposed in the second end region 970 (e.g., in the reservoir 969). The bodily fluids collected in the fluid collection assembly 952 may be removed from the chamber 958 via the assembly tube 964.

In some examples, the inlet of the assembly tube 964 may not extend into the reservoir 969. In such examples, the inlet of the assembly tube 964 may be disposed within the porous material 956 (fluid permeable membrane 966 and/or fluid permeable support 968) or at a terminal end thereof. For example, an end of the assembly tube 964 may be coextensive with or recessed within the fluid permeable membrane 966 and/or fluid permeable support 968.

Locating the inlet of the assembly tube 964 at or near a location expected to be the gravimetrically low point of the chamber 958 when worn by a patient enables the assembly tube 964 to receive more of the bodily fluids than if inlet of the assembly tube 964 was located elsewhere and reduce the likelihood of pooling (e.g., pooling of the bodily fluids may cause microbe growth and foul odors). For instance, the bodily fluids in the fluid permeable membrane 966 and the fluid permeable support 968 may flow in any direction due to capillary forces. However, the bodily fluids may exhibit a preference to flow in the direction of gravity, especially when at least a portion of the fluid permeable membrane 966 and/or the fluid permeable support 968 is saturated with the bodily fluids. Accordingly, one or more of the inlet of the assembly tube 964 or the reservoir 969 may be located in the fluid collection assembly 952 in a position expected to be the gravimetrically low point in the fluid collection assembly 952 when worn by a patient, such as the second end region 972.

In an example, the assembly tube 964 is configured to be at least insertable into the chamber 958. In such an example, the assembly tube 964 may include one or more markers (not shown) on an exterior thereof that are located to facilitate insertion of the assembly tube 964 into the chamber 958. For example, the assembly tube 964 may include one or more markings thereon that are configured to prevent over or under insertion of the assembly tube 964, such as when the assembly tube 964 defines an inlet of the assembly tube 964 that is configured to be disposed in or adjacent to the reservoir 969. In another example, the assembly tube 964 may include one or more markings thereon that are configured to facilitate correct rotation of the assembly tube 964 relative to the chamber 958. The one or more markings may include a line, a dot, a sticker, or any other suitable marking.

The assembly tube 964 may exhibit a generally circular cross-sectional shape or exhibit another cross-sectional shape exhibiting a width that is not significantly greater than the thickness thereof, wherein the width and thickness are measured perpendicular to a longitudinal axis of the assembly tube 964. The width and thickness of the assembly tube 964 may be about 4 mm or greater, about 5 mm or greater, about 5 mm or greater, about 7 mm or greater, about 8 mm or greater, about 10 mm or greater, about 12 mm or greater, or about 15 mm or greater. The conduit defined by the assembly tube 964 may exhibit a cross-sectional area that is about 15 mm² or greater, about 20 mm² or greater, about 25 mm² or greater, about 30 mm² or greater, about 35 mm² or greater, about 40 mm² or greater, about 50 mm² or greater, or in ranges of about 15 mm² to about 25 mm², about 20 mm² to about 30 mm², about 25 mm² to about 35 mm², about 30 mm² to about 40 mm², or about 35 mm² to about 50 mm².

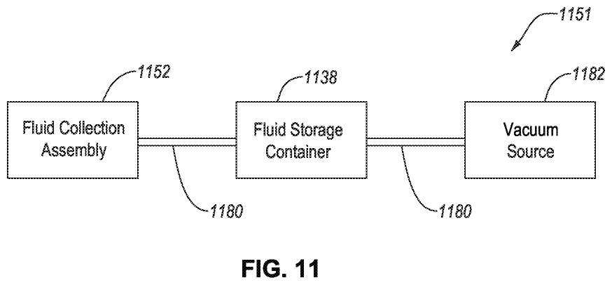

The fluid collection assembly 952 may be coupled to a tube 900 that is distinct from the assembly tube 964. The tube 900 may be the same or substantially similar to any of the tubes having an elongated cross-section that are disclosed herein. The inlet 902 of the tube 900 may be attached to the assembly tube 964 (e.g., an outlet of the assembly tube 964, as shown) or the fluid outlet 965. The tube 900 is configured to fluidly couple the vacuum source (not shown) to the chamber 958 (e.g., the reservoir 969). As the vacuum source (FIG. 11) applies a vacuum/suction in the assembly tube 964, the bodily fluids in the chamber 958 (e.g., at the second end region 970 such as in the reservoir 969) may be drawn into assembly tube 964 and out of the fluid collection assembly 952 via the tube 900.

The tubes disclosed herein may also be used with a male fluid collection assembly that is configured to collect bodily fluids from a male urethral opening (e.g., penis). FIG. 10A is an isometric view of a portion of a fluid collection system 1051 that includes a male fluid collection assembly 1052 and a tube 1000, according to an embodiment. FIG. 10B is a cross-sectional view of the fluid collection system 1051 taken along plane 10B-10B as shown in FIG. 10A, according to an embodiment. The fluid collection assembly 1052 includes a sheath 1073 and a base 1074. The sheath 1073 includes a fluid impermeable barrier 1054 that is at least partially formed from a first panel 1075 and a second panel 1076. The first panel 1075 and the second panel 1076 may be attached or integrally formed together (e.g., exhibits single piece construction). In an embodiment, as illustrated, the first panel 1075 and the second panel 1076 are distinct sheets. The fluid impermeable barrier 1054 also defines a chamber 1058 between the first panel 1075 and the second panel 1076, an opening 1060 at a first end region 1070 of the sheath 1073, and an fluid outlet 1065 at a second end region 1072 of the sheath 1073. The sheath 1073 also includes at least one porous material 1056 disposed in the chamber 1058. The base 1074 includes an aperture 1078. The base 1074 is permanently attached to the first end region 1070 of the sheath 1073 such that the aperture 1078 is aligned with the opening 1060. Permanently attached means that the sheath 1073 cannot be detached from the base 1074 without damaging at least one of the sheath 1073 or the base 1074, using a blade to separate the sheath 1073 from the base 1074, and/or using chemicals to dissolve the adhesive that attaches the sheath 1073 from the base 1074.

The inner surfaces of the fluid impermeable barrier 1054 (e.g., inner surfaces of the first and second panels 1075, 1001076 at least partially defines the chamber 1058 within the fluid collection assembly 1052. The fluid impermeable barrier 1054 temporarily stores the bodily fluids in the chamber 1058. The fluid impermeable barrier 1054 may be formed of any of the fluid impermeable materials disclosed herein. At least one or more portions of at least an outer surface 1062 of the fluid impermeable barrier 1054 may be formed from a soft and/or smooth material, thereby reducing chaffing.

In an embodiment, at least one of the first panel 1075 or the second panel 1076 is formed from an at least partially transparent fluid impermeable material, such as polyethylene, polypropylene, polycarbonate, or polyvinyl chloride. Forming at least one of the first panel 1075 or the second panel 1076 from an at least partially transparent fluid impermeable material allows a person (e.g., medical practitioner) to examiner the penis. In some embodiments, both the first panel 1075 and the second panel 1076 are formed from at least partially transparent fluid impermeable material. Selecting at least one of the first panel 1075 or the second panel 1076 to be formed from an at least partially transparent impermeable material allows the penis to be examined without detaching the entire fluid collection assembly 1052 from the region about the penis. For example, the chamber 1058 may include a penis receiving area 1077 that is configured to receive the penis of the individual when the penis extends into the chamber 1058. The penis receiving area 1077 may be defined by at least the porous material 1056 and at least a portion of the at least partially transparent material of the first panel 1075 and/or the second panel 1076. In other words, the porous material 1056 is positioned in the chamber 1058 such that the porous material 1056 is not positioned between the penis and at least a portion of the transparent portion of the first panel 1075 and/or second panel 1076 when the penis is inserted into the chamber 1058 through the opening 1060. The porous material 1056 is generally not transparent and, thus, the portion of the at least partially transparent material of the first panel 1075 and/or the second panel 1076 that defines the penis receiving area 1077 forms a window which allows the person to view into the penis receiving area 1077 and examine the penis.

The opening 1060 defined by the fluid impermeable barrier 1054 provides an ingress route for fluids to enter the chamber 1058 when the penis is a buried penis and allow the penis to enter the chamber 1058 (e.g., the penis receiving area 1077) when the penis is not buried. The opening 1060 may be defined by the fluid impermeable barrier 1054 (e.g., an inner edge of the fluid impermeable barrier 1054). For example, the opening 1060 is formed in and extends through the fluid impermeable barrier 1054 thereby enabling bodily fluids to enter the chamber 1058 from outside of the fluid collection assembly 1052.

The fluid impermeable barrier 1054 defines an fluid outlet 1065 sized to receive an assembly tube 1064. The assembly tube 1064 may be at least partially disposed in the chamber 1058 or otherwise in fluid communication with the chamber 1058 through the fluid outlet 1065. The fluid outlet 1065 may be sized and shaped to form an at least substantially fluid tight seal against the assembly tube 1064 thereby substantially preventing the bodily fluids from escaping the chamber 1058. In an embodiment, the fluid outlet 1065 may be formed from a portion of the first panel 1075 and the second panel 1076 that are not attached or integrally formed together. In such an embodiment, the fluid impermeable barrier 1054 may not include a cap exhibiting a rigidity that is greater than the portions of the fluid impermeable barrier 1054 thereabout which may facilitate manufacturing of the fluid collection assembly 1052 may decreasing the number of parts that are used to form the fluid collection assembly 1052 and may decrease the time required to manufacture the fluid collection assembly 1052. The lack of the cap may make securing the assembly tube 1064 to the fluid outlet 1065 using interference fit to be difficult though, it is noted, attaching the assembly tube 1064 to the fluid outlet 1065 may still be possible. As such, the assembly tube 1064 may be attached to the fluid outlet 1065 (e.g., to the first and second panels 1075, 1076) using an adhesive, a weld, or otherwise bonding the fluid outlet 1065 to the fluid outlet 1065. Attaching the assembly tube 1064 to the fluid outlet 1065 may prevent leaks and may prevent the assembly tube 1064 from inadvertently becoming detached from the fluid outlet 1065. In an example, the assembly tube 1064 may be attached to the fluid outlet 1065 in the same manufacturing step that attaches the first and second panels 1075, 1076 together.

As previously discussed, the sheath 1073 includes at least one porous material 1056 disclosed in the chamber 1058. The porous material 1056 may direct the bodily fluids to one or more selected regions of the chamber 1058, such as away from the penis and towards the fluid outlet 1065. The porous material 1056 may include any of the porous materials disclosed herein, such as any of the wicking materials disclosed herein.

In an embodiment, the porous material 1056 may be a sheet. Forming the porous material 1056 as a sheet may facilitate the manufacturing of the fluid collection assembly 1052. For example, forming the porous material 1056 as a sheet allows the first panel 1075, the second panel 1076, and the porous material 1056 to each be sheets. During the manufacturing of the fluid collection assembly 1052, the first panel 1075, the second panel 1076, and the porous material 1056 may be stacked and then attached to each other in the same manufacturing step. For instance, the porous material 1056 may exhibit a shape that is the same size or, more preferably, slightly smaller than the size of the first panel 1075 and the second panel 1076. As such, attaching the first panel 1075 and the second panel 1076 together along the outer edges 134 thereof may also attach the porous material 1056 to the first panel 1075 and the second panel 1076. The porous material 1056 may be slightly smaller than the first panel 1075 and the second panel 1076 such that the first panel 1075 and/or the second panel 1076 extend around the porous material 1056 such that the porous material 1056 does not form a passageway through the fluid impermeable barrier 1054 through which the bodily fluids may leak. Also, attaching the porous material 1056 to the first panel 1075 and/or the second panel 1076 may prevent the porous material 1056 from significantly moving in the chamber 1058, such as preventing the porous material 1056 from bunching together near the fluid outlet 1065. In an example, the porous material 1056 may be attached to the first panel 1075 or the second panel 1076 (e.g., via an adhesive) before or after attaching the first panel 1075 to the second panel 1076. In an example, the porous material 1056 may merely be disposed in the chamber 1058 without attaching the porous material 1056 to at least one of the first panel 1075 or the second panel 1076. In an embodiment, as will be discussed in more detail below, the porous material 1056 may exhibit shapes other than a sheet, such as a hollow generally cylindrical shape.

Generally, the sheath 1073 is substantially flat when the penis is not in the penis receiving area 1077 and the sheath 1073 is resting on a flat surface. The sheath 1073 is substantially flat because the fluid impermeable barrier 1054 is formed from the first panel 1075 and the second panel 1076 instead of a generally tubular fluid impermeable barrier. Further, as previously discussed, the porous material 1056 may be a sheet, which also causes the sheath 1073 to be substantially flat. The sheath 1073 may also be substantially flat because the fluid collection assembly 1052 may not include relatively rigid rings or caps that exhibit a rigidity that is greater than the portions of the fluid impermeable barrier 1054 thereabout since such rings and caps may inhibit the sheath 1073 being substantially flat. It is noted that the sheath 1073 is described as being substantially flat because at least one of the porous material 1056 may cause a slight bulge to form in the sheath 1073 depending on the thickness of the porous material 1056, the fluid outlet 1065 and/or assembly tube 1064 may cause a bulge thereabout, or the base 1074 may pull on portions of the sheath 1073 thereabout. It is also noted that the sheath 1073 may also be compliant and, as such, the sheath 1073 may not be substantially flat during use since, during use, the sheath 1073 may rest on a non-flat surface (e.g., may rest on the testicles, the perineum, and/or between the thighs) and the sheath 1073 may conform to the surface of these shapes.

The ability of the sheath 1073 to be substantially flat when the penis is not in the penis receiving area 1077 and the sheath 1073 is resting on a flat surface allows the fluid collection assembly 1052 to be used with a buried and a non-buried penis. For example, when the fluid collection assembly 1052 is being used with a buried penis, the penis does not extend into the penis receiving area 1077 which causes the sheath 1073 to lie relatively flat across the aperture 1078. When the sheath 1073 lies relatively flat across the aperture 1078, the porous material 1056 extends across the aperture 1078 and is in close proximity to the buried penis. As such, the porous material 1056 prevents or inhibits pooling of bodily fluids discharged from the buried penis against the skin of the individual since the porous material 1056 will receive and remove at least a significant portion of the bodily fluids that would otherwise pool against the skin of the individual. Thus, the skin of the individual remains dry thereby improving comfort of using the fluid collection assembly 1052 and preventing skin degradation. However, unlike other conventional fluid collection assemblies that are configured to be used with buried penises, the fluid collection assembly 1052 may still be used with a non-buried penis since the non-buried penis can still be received into the penis receiving area 1077, even when the penis is fully erect. Additionally, the ability of the sheath 1073 to be substantially flat allows the fluid collection assembly 1052 to be used more discretely than if the sheath 1073 was not substantially flat thereby avoiding possibly embarrassing scenarios.

When the sheath 1073 is substantially flat, the porous material 1056 occupies substantially all of the chamber 1058 and the penis receiving area 1077 is collapsed (shown as being non-collapsed in FIG. 10B for illustrative purposes). In other words, the sheath 1073 may not define an region that is constantly unoccupied by the porous material 1056. When the porous material 1056 occupies substantially all of the chamber 1058, the bodily fluids discharged into the chamber 1058 are unlikely to pool for significant periods of time since pooling of the bodily fluids may cause sanitation issues, cause an odor, and/or may cause the skin of the individual to remain in contact with the bodily fluids which may cause discomfort and skin degradation.

As previously discussed, the first panel 1075, the second panel 1076, and the porous material 1056 may be selected to be relatively flexible. The first panel 1075, the second panel 1076, and the porous material 1056 are relatively flexible when the first panel 1075, the second panel 1076, and the porous material 1056, respectively, are unable to maintain their shape when unsupported. The flexibility of the first panel 1075, the second panel 1076, and the porous material 1056 may allow the sheath 1073 to be substantially flat, as discussed above. The flexibility of the first panel 1075, the second panel 1076, and the porous material 1056 may also allow the sheath 1073 to conform to the shape of the penis even when the size and shape of the penis changes (e.g., becomes erect) and to minimize any unoccupied spaces in the chamber 1058 in which bodily fluids may pool.

As previously discussed, the fluid collection assembly 1052 includes a base 1074 that is configured to be permanently attached to the sheath 1073. The base 1074 is configured to be permanently attached to the sheath 1073 when, for example, when the fluid collection assembly 1052 is provided with the base 1074 permanently attached to the sheath 1073 or the base 1074 is provided without being permanently attached to the sheath 1073 but is configured to be permanently attached to the sheath 1073 at some point in the future. The base 1074 may be permanently attached to the sheath 1073 using any suitable technique. For example, the base 1074 may be permanently attached to the sheath 1073 using an adhesive, sewing, heat sealing, RF welding, or US welding.

As previously discussed, the base 1074 is sized, shaped, and made of a material to be coupled to the skin that surrounds the penis (e.g., mons pubis, thighs, testicles, and/or perineum) and have the penis disposed therethrough. For example, the base 1074 may define an aperture 1078 configured to have the penis positioned therethrough. In an example, the base 1074 may exhibit the general shape or contours of the skin surface that the base 1074 is configured to be coupled with. The base 1074 may be flexible, thereby allowing the base 1074 to conform to any shape of the skin surface and mitigate the base 1074 pulling the on skin surface. The base 1074 may extend laterally past the sheath 1073 thereby increasing the surface area of the skin of the individual to which the fluid collection assembly 1052 may be attached compared to a substantially similar fluid collection assembly 1052 that did not include a base.

As previously discussed, the fluid collection assembly 1052 includes an assembly tube 1064. The assembly tube 1064 may be the same or substantially similar to any of the assembly tubes disclosed herein. An inlet of the assembly tube 1064 may be located at or near the first end region 1070 of the sheath 1073 which is expected to be the gravimetrically low point of the chamber 1058 when worn by a user. Locating the inlet of the assembly tube 1064 at or near the first end region 1070 of the sheath 1073 enables the assembly tube 1064 to receive more of the bodily fluids than if the inlet of the assembly tube 1064 was located elsewhere and reduce the likelihood of pooling (e.g., pooling of the bodily fluids may cause microbe growth and foul odors). For instance, the bodily fluids in porous material 1056 due to capillary forces. However, the bodily fluids may exhibit a preference to flow in the direction of gravity, especially when at least a portion of the porous material 1056 is saturated with the bodily fluids. Accordingly, the inlet of the assembly tube 1064 may be located in the fluid collection assembly 1052 in a position expected to be the gravimetrically low point in the fluid collection assembly 1052 when worn by a user.

In an example, the assembly tube 1064 is configured to be at least insertable into the chamber 1058, such as into the penis receiving area 1077. In such an example, the assembly tube 1064 may include one or more markers (not shown) on an exterior thereof that are located to facilitate insertion of the assembly tube 1064 into the chamber 1058. For example, the assembly tube 1064 may include one or more markings thereon that are configured to prevent over or under insertion of the assembly tube 1064. In another example, the assembly tube 1064 may include one or more markings thereon that are configured to facilitate correct rotation of the assembly tube 1064 relative to the chamber 1058. The one or more markings may include a line, a dot, a sticker, or any other suitable marking.

Further examples of male fluid collection assemblies are disclosed in U.S. Provisional Patent Application No. 63/067,542 filed on Aug. 19, 2020 and U.S. patent application Ser. No. 16/433,773 filed on Jun. 5, 2019, the disclosures of each of which are incorporated herein, in their entireties, by this reference.

The fluid collection assembly 1052 may be coupled to a tube 1000 that is distinct from the assembly tube 1064. The tube 1000 may be the same or substantially similar to any of the tubes having an elongated cross-section that are disclosed herein. The tube 1000 may be attached to the assembly tube 1064 (e.g., an outlet of the assembly tube 1064, as shown) or the fluid outlet 1065. The tube 1000 is configured to fluidly couple the vacuum source (not shown) to the chamber 1058 (e.g., the reservoir 1069). As the vacuum source (FIG. 11) applies a vacuum/suction in the assembly tube 1064, the bodily fluids in the chamber 1058 (e.g., at the second end region 1072) may be drawn into assembly tube 1064 and out of the fluid collection assembly 1052 via the tube 1000.

The fluid collection assembly that are attached, either directly or indirectly, to the tubes disclosed herein may include fluid collection assemblies other than the female and male fluid collection assemblies disclosed herein. In an example, the fluid collection assembly may include a gender neutral fluid collection assembly, such as a Foley catheter.

In the embodiments discussed with regards to FIGS. 9A-10B, the inlets of the tubes 900, 1000 are configured to be attached to the fluid outlets of the fluid collection assemblies and/or the assembly tubes. For example, the inlets of the tubes 900, 1000 do not exhibit an elongated cross-section (e.g., exhibit a generally circular cross-section) such that the tubes 900, 1000 may be attached to the fluid outlets and/or assembly tubes. However, in other embodiments, at least one of the fluid outlets and/or the assembly tubes may be configured to be attached to the inlets of the tubes 900, 1000 when the inlets exhibit an elongated cross-section, similar to the inlet 102 of FIG. 1A. In such embodiments, the fluid outlets and/or the assembly tubes may exhibit an elongated cross-section and/or perimeter that corresponds to (e.g., is slightly larger than, the same as, or smaller than) the elongated cross-section and/or perimeter of the inlet of the tubes 900, 1000.

FIG. 11 is a block diagram of a fluid collection system 1151, according to an embodiment. The system 1151 includes a fluid collection assembly 1152 and a fluid storage container 1138. The fluid collection assembly 1152 and the fluid storage container 1138 may be fluidly coupled to each other via one or more tubes 1180. The one or more tubes 1180 include any of the tubes with an elongated cross-section, such as one or more of the tubes 100, 200, 300, 400, 500, 600, 700, 800, 900a, 900b, or 1000. The one or more tubes 1180 may also include one or more conventional tubes, such as an assembly tube. The fluid collection system 1151 may also include a vacuum source 1182, for example, when the fluid collection assembly 1152 includes a female or male fluid collection assembly.

Bodily fluids (e.g., urine) collected in the fluid collection assembly 1152 may be removed from the fluid collection assembly 1152 via the tubes 1180 which protrudes into the fluid collection assembly 1152. For example, an inlet of the tubes 1180 may extend into the fluid collection assembly 1152, such as to a reservoir therein. The outlet of the tubes 1180 may extend into the fluid storage container 1138 or the vacuum source 1182. Suction force may be introduced into the chamber of the fluid collection assembly 1152 via the inlet of the tubes 1180 responsive to suction (e.g., vacuum) force applied at the outlet of the tubes 1180.

The suction force may be applied to the outlet of the tubes 1180 by the vacuum source 1182 either directly or indirectly. The suction force may be applied indirectly via the fluid storage container 1138. For example, the outlet of the tubes 1180 may be disposed within the fluid storage container 1138 and an additional tubes 1180 may extend from the fluid storage container 1138 to the vacuum source 1182. Accordingly, the vacuum source 1182 may apply suction to the fluid collection assembly 1152 via the fluid storage container 1138. The suction force may be applied directly via the vacuum source 1182. For example, the outlet of the tubes 1180 may be disposed within the vacuum source 1182. An additional tubes 1180 may extend from the vacuum source 1182 to a point outside of the fluid collection assembly 1152, such as to the fluid storage container 1138. In such examples, the vacuum source 1182 may be disposed between the fluid collection assembly 1152 and the fluid storage container 1138.

The fluid storage container 1138 is sized and shaped to retain the bodily fluids therein. The fluid storage container 1138 may include a bag (e.g., drainage bag), a bottle or cup (e.g., collection jar), or any other enclosed container for storing the bodily fluids such as urine. In some examples, the tubes 1180 may extend from the fluid collection assembly 1152 and attach to the fluid storage container 1138 at a first point therein. An additional tubes 1180 may attach to the fluid storage container 1138 at a second point thereon and may extend and attach to the vacuum source 1182. Accordingly, a vacuum (e.g., suction) may be drawn through fluid collection assembly 1152 via the fluid storage container 1138. Fluid, such as urine, may be drained from the fluid collection assembly 1152 using the vacuum source 1182.

The vacuum source 1182 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 1182 may provide a vacuum or suction to remove fluid from the fluid collection assembly 1152. In some examples, the vacuum source 1182 may be powered by one or more of a power cord (e.g., connected to a power socket), one or more batteries, or even manual power (e.g., a hand operated vacuum pump). In some examples, the vacuum source 1182 may be sized and shaped to fit outside of, on, or within the fluid collection assembly 1152. For example, the vacuum source 1182 may include one or more miniaturized pumps or one or more micro pumps. The vacuum sources 1182 disclosed herein may include one or more of a switch, a button, a plug, a remote, or any other device suitable to activate the vacuum source 1182.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A tube configured to be connected to a fluid collection assembly, the tube comprising:
an inlet exhibiting an inlet width and an inlet thickness that are each measured perpendicular to a longitudinal axis of the tube, wherein the inlet width is at most about 4 times greater than the inlet thickness, wherein the inlet width and the inlet thickness are a maximum width and a maximum thickness of the inlet, respectively, when the tube is positioned on a surface with no external forces applied to the inlet;
an outlet downstream from the inlet, the outlet exhibiting an outlet width and an outlet thickness that are each measured perpendicular to the longitudinal axis of the tube, wherein the outlet width and the outlet thickness are a maximum width and a maximum thickness of the outlet, respectively, when the tube is positioned on a surface with no external forces applied to the outlet; and
an intermediate portion between the inlet and the outlet, the intermediate portion exhibiting an intermediate width and an intermediate thickness that are each measured perpendicular to the longitudinal axis of the tube, the intermediate width at least about 5 times greater than the intermediate thickness, wherein the intermediate width and the intermediate thickness are a maximum width and a maximum thickness of the intermediate portion, respectively, when the tube is positioned on a surface with no external forces applied to the outlet;
wherein the inlet, the outlet, and the intermediate portion define a conduit extending from the inlet to the outlet.

2. The tube of claim 1, wherein the inlet width is substantially equal to the inlet thickness.

3. The tube of a claim 1, further comprising a tapered portion extending from and between the inlet and the intermediate portion, the tapered portion exhibiting at least one of a width or a thickness that varies from the inlet to the intermediate portion.

4. The tube of claim 3, wherein the inlet, the tapered portion, and the intermediate portion exhibit single piece construction.

5. The tube of claim 3, further comprising a first piece and a second piece that is distinct from the first piece, the first piece including the inlet and at least a portion of the tapered portion and the second piece including at least a portion of the intermediate portion.

6. The tube of claim 5, wherein the first piece is at least one of more rigid or thicker than the first piece.

7. The tube of claim 1, wherein the outlet width is at most about 4 times greater than the outlet thickness.

8. (canceled)

9. (canceled)

10. The tube of claim 1, wherein the intermediate width is at least about 10 times greater than the intermediate thickness.

11. The tube of claim 1, wherein the intermediate width is at least about 25 times greater than the intermediate thickness.

12. The tube of claim 1, further comprising one or more fluid flow features disposed in the conduit, the one or more fluid flow features defining a plurality of channels through which one or more fluids may flow.

13. The tube of claim 12, wherein the one or more fluid flow features a base and a plurality of walls extending from the base, the base and the plurality of walls at least partially defining the plurality of channels.

14. The tube of a claim 12, wherein the one or more fluid flow features includes a plurality of pipes, each of the plurality of pipes defining at least one of the plurality of channels.

15. The tube of claim 12, wherein the one or more fluid flow features includes a porous material defining a plurality of pores, the plurality of pores forming the plurality of channels.

16. The tube of claim 12, wherein the cross-sectional area of the plurality of channels is at least about 30 mm² or greater.

17. The tube of claim 12, wherein at least one of the plurality of channels includes an entrance and an exit downstream from the entrance, wherein a width of the at least one of the plurality of channels decreases along at least a portion of a length thereof in a direction extending from the entrance to the exit.

18. The tube of a claim 1, further comprising a disinfectant coating on at least a portion of a surface defining the conduit that is proximate to the inlet.

19. A fluid collection system, comprising:
a fluid collection assembly defining a fluid outlet;
a fluid storage container defining a fluid inlet; and
at least one tube configured to be connected to the fluid collection assembly, the at least one tube comprising:
an inlet exhibiting an inlet width and an inlet thickness that are each measured perpendicular to a longitudinal axis of the tube, wherein the inlet width is at most about 4 times greater than the inlet thickness, wherein the inlet width and the inlet thickness are a maximum width and a maximum thickness of the inlet, respectively, when the tube is positioned on a surface with no external forces applied to the inlet;
an outlet downstream from the inlet downstream from the inlet, the outlet exhibiting an outlet width and an outlet thickness that are each measured perpendicular to the longitudinal axis of the tube, wherein the outlet width and the outlet thickness are a maximum width and a maximum thickness of the outlet, respectively, when the tube is positioned on a surface with no external forces applied to the outlet; and
an intermediate portion between the inlet and the outlet, the intermediate portion exhibiting an intermediate width and an intermediate thickness that are each measured perpendicular to the longitudinal axis of the tube, the intermediate width at least about 5 times greater than the intermediate thickness, wherein the intermediate width and the intermediate thickness are a maximum width and a maximum thickness of the intermediate portion, respectively, when the tube is positioned on a surface with no external forces applied to the outlet;
wherein the inlet, the outlet, and the intermediate portion define a conduit extending from the inlet to the outlet.

20. (canceled)

21. (canceled)

22. (canceled)

23. The fluid collection system of claim 19, further comprising at least one additional tube directly attached to and extending between the fluid outlet of the fluid collection assembly and the inlet of the at least one tube.

24. (canceled)

25. A method of using a fluid collection system, the method comprising:
receiving one or more bodily fluids from a patient with a fluid collection assembly, the fluid collection assembly including a fluid outlet;
flowing the one or more bodily fluids out of the fluid outlet, into an inlet of a tube, through an intermediate portion of the tube, and out an outlet of the tube, wherein:
the inlet exhibits an inlet width and an inlet thickness that are each measured perpendicular to a longitudinal axis of the tube, the inlet width at most about 4 times greater than the inlet thickness, wherein the inlet width and the inlet thickness are a maximum width and a maximum thickness of the inlet, respectively, when the tube is positioned on a surface with no external forces applied to the inlet;
the outlet located downstream from the inlet, the outlet exhibiting an outlet width and an outlet thickness that are each measured perpendicular to the longitudinal axis of the tube, wherein the outlet width and the outlet thickness are a maximum width and a maximum thickness of the outlet, respectively, when the tube is positioned on a surface with no external forces applied to the outlet; and
the intermediate portion between the inlet and the outlet, the intermediate portion exhibiting an intermediate width and an intermediate thickness that are each measured perpendicular to the longitudinal axis of the tube, the intermediate width at least about 5 times greater than the intermediate thickness, wherein the intermediate width and the intermediate thickness are a maximum width and a maximum thickness of the intermediate portion, respectively, when the tube is positioned on a surface with no external forces applied to the outlet;
the inlet, the outlet, and the intermediate portion define a conduit extending from the inlet to the outlet.

26. The method of claim 25, further comprising positioning the tube underneath the patient.
